(19) United States
(12) Patent Application Publication
(10) Pub. No.: US 2010/0261908 A1
(43) Pub. Date: Oct. 14, 2010

(54) PROCESSES FOR THE PREPARATION OF PRASUGREL , AND ITS SALTS AND POLYMORPHS

(75) Inventors: Pratap Reddy Padi, Hyderabad (IN); Seetha Rama Sarma Peri, Hyderabad (IN); Madhusudhan Reddy Ganta, Hyderabad (IN); Srinivas Polavarapu, Hyderabad (IN); Praveen Cherukupally, Hyderabad (IN); Babu Ireni, Nizamabad District (IN); Shailaja Padamata, Hyderabad (IN); Krishna Jonnada, Hyderabad (IN); Krishna Vinigari, Mahaboobnagar (IN); Kavitha Nerella, Krishna District (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500016 Andhra Pradesh, AP (IN); DR. REDDY'S LABORATORIES, INC., Bridgewater, NJ (US)

(21) Appl. No.: 12/741,671

(22) PCT Filed: Nov. 7, 2008
(86) PCT No.: PCT/US08/82815

§ 371 Date: May 6, 2010

Related U.S. Application Data
(60) Provisional application No. 61/058,601, filed on Jun. 4, 2008.
(60) Provisional application No. 61/073,180, filed on Jun. 17, 2008.

(30) Foreign Application Priority Data
Nov. 9, 2007 (IN) 2576/CHE/2007
Dec. 6, 2007 (IN) 2909/CHE/2007

(51) Int. Cl. C07D 495/04

(52) U.S. Cl. 546/114

(57) ABSTRACT

Processes for the preparation of prasugrel and its pharmaceutically acceptable salts thereof. Also disclosed are polymorphic forms of prasugrel hydrochloride and processes for their preparation.

This application relates to prasugrel and its salts and polymorphs, processes for preparing prasugrel and its salts and polymorphs, and the use of prasugrel and its salts and polymorphs, especially in pharmaceutical compositions. Further, the present application relates to intermediates of prasugrel, processes for preparing intermediates of prasugrel, and the use of such intermediates for preparing prasugrel and its salts and polymorphs.

Prasugrel has chemical names 2-acetoxy-5(α-cyclopropylcarbonyl-2-fluorobenzyl)-4,5,6,7-tetrahydrothieno[3,2-c]pyridine, or 5-[2-cyclopropyl-1-(2-fluorophenyl)-2-oxoethyl]-4,5,6,7-tetrahydrothieno[3,2-c]pyridin-2-yl acetate, and has structural Formula I.

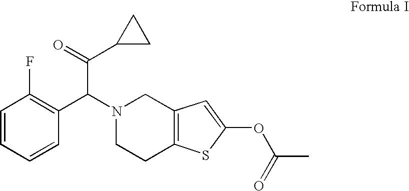

Prasugrel is a member of the thienopyridine class of ADP receptor inhibitors, like ticloidine (TICLID®) and clopidogrel (PLAVIX®). These agents are believed to reduce the aggregation (i.e., clumping) of platelets by irreversibly binding to P2Y12 receptors. Prasugrel is a novel platelet inhibitor that is expected to be administered as a solid oral dosage form. Prasugrel is undergoing the approval process for acute coronary syndromes planned for percutaneous coronary intervention (PCI).

U.S. Pat. No. 5,288,726 discloses prasugrel and pharmaceutically acceptable salt thereof, and their use in the treatment or prophylaxis of thrombosis or embolisms. It also discloses a process for the preparation of prasugrel. It discloses the reaction of 2-fluorobenzyl bromide with cyclopropyl cyanide in the presence of magnesium and ether to provide cyclopropyl 2-fluorobenzyl ketone of Formula 1-V, which is then reacted with bromine in presence of carbon tetrachloride to provide 2-fluorobenzylcarbonyl bromide of Formula 1-II. Condensation of compound of Formula 1-II with 5,6,7,7a-tetrahydro-thieno[3,2-c]pyridin-2(4H)-one hydrochloride of Formula 1-IIIB in the presence of anhydrous potassium carbonate in DMF affords 5-[2-cyclopropyl-1-(2-fluorophenyl)-2-oxoethyl]-5,6,7,7a-tetrahydro-thieno[3,2-c]pyridin-2(4H)-one of Formula 1-1V, which is finally reacted with acetic anhydride and sodium hydride in DMF followed by column chromatography purification to yield prasugrel of Formula I. The process is schematically represented in Scheme 1.

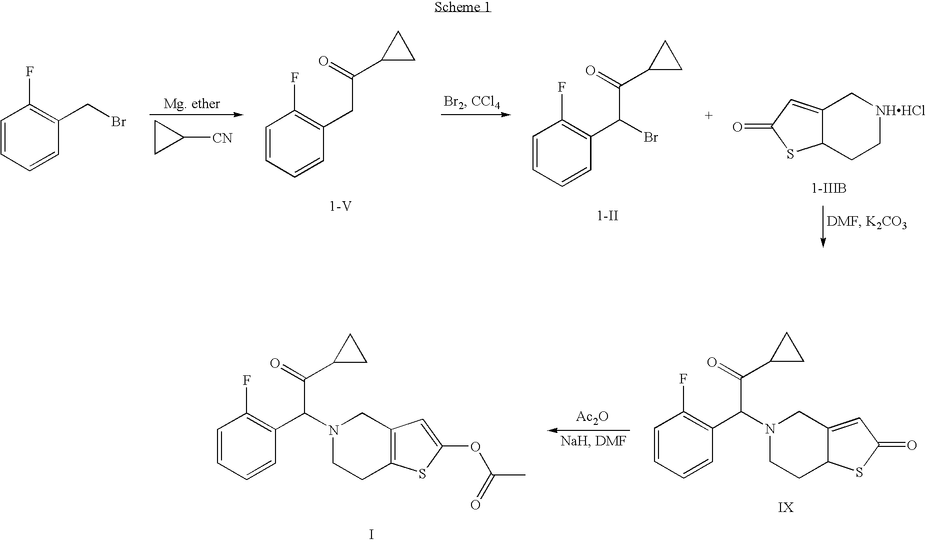

U.S. Pat. No. 5,874,581 discloses a process for preparing prasugrel. The process disclosed for the preparation of 2-(tert-butyldimethylsilyloxy)-4,5,6,7-tetrahydro-thieno[3,2-c]pyridine involves the reaction of 4,5,6,7-tetrahydrothieno[3,2-c]pyridin-2(3H)-one p-toluenesulfonate with tert-butyldimethylsilyl chloride (TBDMS-Cl) in presence of triethylamine. The disclosed process involves the reaction of 2-fluorophenyl acetic acid with ethyl cyclopropane carboxylate in presence of isopropyl magnesium bromide to provide cyclopropyl 2-fluorobenzyl ketone, which is reacted with sulfuryl chloride to yield 2-fluoro-α-cyclopropyl carbonyl benzyl chloride of Formula 2-IIA. Condensation of compound of Formula 2-IIB with compound of Formula 2-IIA in presence of triethylamine to yield 2-(tert-butyldimethylsilyloxy)-5-[2-cyclopropyl-1-(2-fluorophenyl)-2-oxoethyl]5,6,7,7a-tetrahydro-thieno[3,2-c]pyridine of Formula 2-IIC, which is finally reacted with acetic anhydride in presence of triethyl amine and dimethyl amino pyridine (DMAP) to yield prasugrel of Formula I, as shown in Scheme 2.

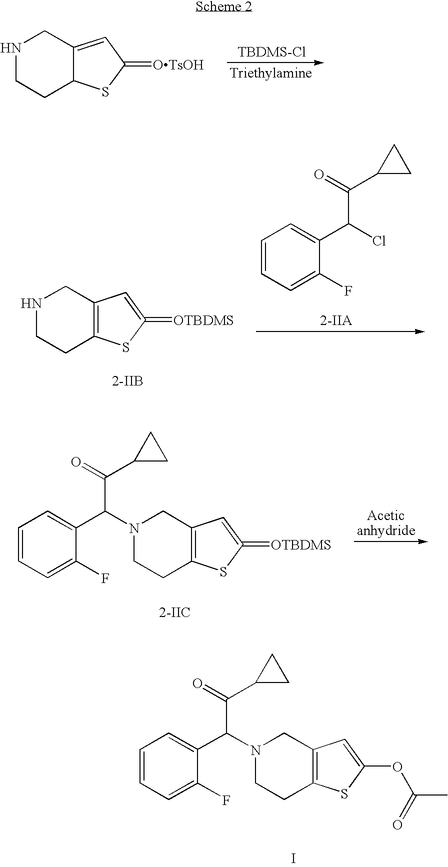

U.S. Pat. No. 4,740,510 discloses a process for the preparation of an intermediate 5,6,7,7A-tetrahydro-thieno[3,2-C]pyridin-2(4H)-one of Formula 3-IIIB as shown in Scheme 3.

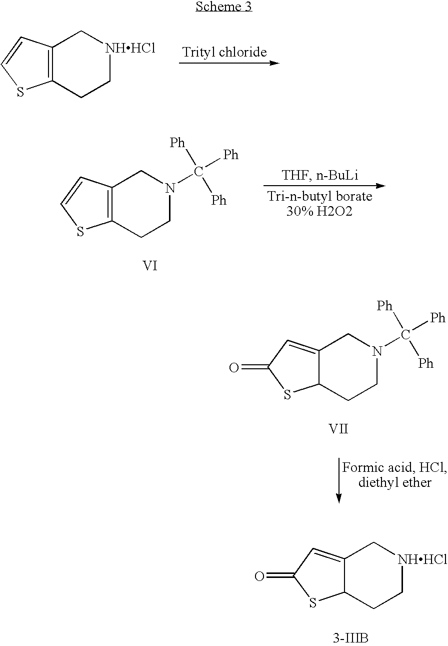

International Application Publication No. WO 2007/114526 A1 discloses the process for the preparation of high purity prasugrel or its hydrochloride salt having reduced OXTP content. OXTP is the compound of Formula IV or its enol form of thienopyridine ring.

International Application Publication No. WO 2008/108291 A1 discloses a process for the preparation of prasugrel hydrochloride having reduced CATP content. The structure of the CATP impurity can be structurally represented by the formula below.

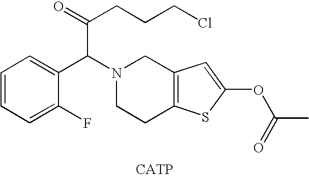

The above disclosed processes have certain drawbacks. These processes either involve time-consuming processes, such as, for example, column purifications or tedious separations to reduce the impurities level. These processes involve the use of high solvent quantities. Hence, there remains a need for viable processes to prepare prasugrel or its salts.

U.S. Pat. No. 6,693,115 discloses and claims prasugrel hydrochloride and prasugrel maleate. Further, it discloses three crystal forms for prasugrel hydrochloride, designated as crystal A, crystal B1, and crystal B2. Nevertheless, there is a continuing need for new polymorphic forms of prasugrel hydrochloride and processes for preparation thereof.

### SUMMARY

The present invention includes processes for preparing prasugrel or its pharmaceutically acceptable salts.

The present invention also includes processes for purifying prasugrel or its pharmaceutically acceptable salts.

The present invention also includes new intermediates designated as compounds of Formula III and Formula VIII,

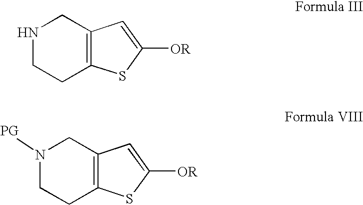

wherein R is a hydroxyl-protecting group and PG is a nitrogen-protecting group.

The present invention includes novel polymorphic and amorphous forms of prasugrel hydrochloride and processes for their preparation.

The present invention includes processes for the conversion of weak acid addition salt of prasugrel to a strong acid addition salt of prasugrel.

The present invention includes pharmaceutical compositions that comprise a therapeutically effective amount of prasugrel or its pharmaceutically acceptable salt and at least one pharmaceutically acceptable excipient.

### BRIEF DESCRIPTION OF THE DRAWINGS

FIG. 1: X-ray powder diffraction (XRD) pattern of prasugrel hydrochloride amorphous form of the present application.

FIG. 2: Infrared absorption (1R) spectrum of prasugrel hydrochloride amorphous form of the present application.

FIG. 3: X-ray powder diffraction (XRD) pattern of prasugrel hydrochloride Form C of the present application.

FIG. 4: Infrared absorption spectrum of prasugrel hydrochloride Form C of the present application.

FIG. 5: Differential scanning calorimetry (“DSC”) curve of prasugrel hydrochloride Form C of the present application.

FIG. 6: X-ray powder diffraction (XRD) pattern of prasugrel hydrochloride Form D of the present application.

FIG. 7: Infrared absorption spectrum of prasugrel hydrochloride Form D of the present application.

FIG. 8: Differential scanning calorimetry (“DSC”) curve of prasugrel hydrochloride Form D of the present application.

FIG. 9: X-ray powder diffraction (XRD) pattern of prasugrel hydrochloride Form E of the present application.

FIG. 10: Infrared absorption spectrum of prasugrel hydrochloride Form E of the present application.

FIG. 11: Differential scanning calorimetry (“DSC”) curve of prasugrel hydrochloride Form E of the present application.

FIG. 12: X-ray powder diffraction (XRD) pattern of prasugrel crystalline form of Examples 7 and 10.

FIG. 13: Differential scanning calorimetry (“DSC”) curve of prasugrel crystalline form of Examples 7 and 10.

FIG. 14: Thermogravimetric analysis (TGA) curve of prasugrel crystalline form of Examples 7 and 10.

### DETAILED DESCRIPTION

The present invention includes processes for preparing prasugrel or its pharmaceutically acceptable salts by condensing a compound of Formula II

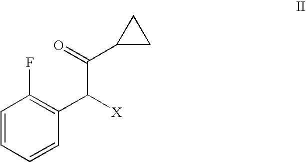

wherein X is halogen, such as, for example, bromine or chlorine, with a compound of Formula III or its acid addition salt,

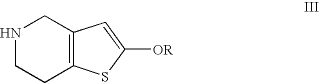

wherein R is acetyl or an hydroxyl-protecting group other than acetyl, to obtain the compound of Formula IV,

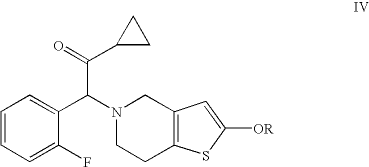

wherein R is as defined above. The intermediate of Formula IV may be subsequently reacted with, e.g., an acetylating agent to obtain prasugrel, which may be optionally converted into a pharmaceutically acceptable salt.

Suitable hydroxyl-protecting groups include and are not limited to tetrahydropyranyl ether, tetrahydrothiopyranyl ether, tetrahydrofuranyl ether, tetrahydrothiofuranyl ether, benzyl carbonate, borate ester, alkyl methyl carbonate, alkylisobutylcarbonate, alkylvinylcarbonate, allyl ether, and cinnamyl ether. Other protecting groups known to a person skilled in the art are all contemplated without limitation.

The process may be schematically represented by Scheme 4.

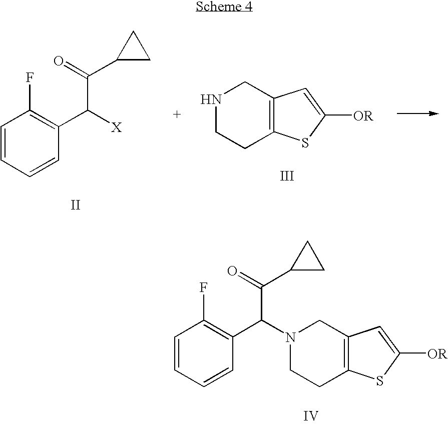

The reaction of a compound of Formula II with a compound of Formula III may be optionally carried out in the presence of a suitable base and/or a solvent.

The reaction may be conducted in the presence or absence of a base. Bases that are useful in the reaction including and are not limited to: inorganic bases, such as, for example, alkali metal or alkaline earth metal carbonates, hydrogen carbonates, hydroxides, oxides, carboxylates, and alkoxides, e.g., sodium carbonate, sodium hydrogen carbonate, potassium carbonate, cesium carbonate, sodium hydroxide, potassium hydroxide, potassium carbonate, potassium t-butoxide, sodium t-butoxide, calcium oxide, sodium acetate, sodium methoxide, and the like; and organic bases, such as, for example, tertiary amines, e.g., triethylamine, N,N-diisopropylethylamine, N,N-diethylethanamine, N-(1-methylethyl)-2-propanamine, 4-ethylmorpholine, 1,4-diazabicyclo[2.2.2]-octane, N-methylmorpholine, pyridine, and the like; or any mixtures thereof.

The solvents that may be utilized for this step include and are not limited to: alcohols, such as, for example, methanol, ethanol, isopropyl alcohol, and n-propanol; halogenated hydrocarbons, such as, for example, dichloromethane, 1,2-dichloroethane, chloroform, and carbon tetrachloride; ketones, such as, for example, acetone, ethylmethyl ketone, and methyl isobutyl ketone; ethers, such as, for example, diethyl ether, dimethyl ether, diisopropyl ether, methyl tertiary-butyl ether, 1,1′-oxybisethane, tetrahydrofuran, and 1,4-dioxane; hydrocarbons, such as, for example, n-heptane, cyclohexane, and n-hexane; aromatic solvents, e.g., benzene, toluene, xylene, chlorobenzene, and methoxybenzene; nitriles, such as, for example, acetonitrile and propionitrile; dimethylsulfoxide (DMSO); N,N-dimethylformamide (DMF); N,N-dimethylacetamide; pyridine; 1,3-dimethyl-3,4,5,6-tetrahydro-2(1H)-pyrimidinone; 1,3-dimethyl-2-imidazolidinone; 1,1,3,3-tetramethylurea; 1-methyl-2-pyrrolidinone; nitrobenzene; water; and mixtures thereof. For example, the solvent(s) may be N,N-dimethylformamide (DMF), dimethylsulfoxide (DMSO), N,N-dimethylacetamide (DMA), dichloromethane, ethylenedichloride, chloroform; and mixtures thereof in various proportions. For example, the solvent is N,N-dimethylformamide (DMF).

The reaction may be carried out for any desired time periods to achieve the desired product yield and purity. Typical reaction times can vary from about 1 hour to 20 hours, or longer. Suitable temperatures for conducting the reaction may range from about 0° C. to about 50° C., or about 20° C. to about 35° C.

Additionally, it may be advantageous to conduct this step under an inert atmosphere, such as, for example, oxygen-free argon or nitrogen gas. The base is used in an amount approximately 1 to 10 moles, or approximately 1 to 5 moles, per mole of compound of Formula II.

After the completion of the reaction, the product may be isolated by filtration followed by washing with water, or the solvent from the reaction may be removed using any suitable method, such as, for example, evaporation, atmospheric distillation, or distillation under vacuum. The organic layer from the reaction mass may be washed with water before distillation.

Distillation of the solvent may be conducted under vacuum, such as, for example, below about 100 mm Hg or below about 600 mm Hg at elevated temperatures, such as, for example, about 20° C. to about 70° C. Any temperature and vacuum conditions may be used as long as they do not influence the nature of the product. The vacuum and the temperature used for the removal of the solvent depend on parameters, such as, for example, the boiling point of the solvent and may be readily determined by persons skilled in the art.

The obtained product may be purified by using column chromatography and recrystallization in suitable solvents.

Optionally, the reaction mass may be diluted by addition of solvent, such as, for example, ethyl acetate, acetone, methyl ethyl ketone, isopropyl alcohol, water, n-hexane, n-heptane, etc., before isolation of the product.

Isolation of the product thus obtained includes collection of the material by any techniques, such as, for example, decantation, filtration by gravity or suction, centrifugation, and the like, and optional washing with solvent. If required, the reaction mass may be cooled before product isolation.

The solid material obtained by any of the techniques described above may be further dried. Drying may be suitably carried out by any methods, such as, for example, use a tray dryer, vacuum oven, air oven, fluidized bed drier, spin flash dryer, flash dryer, and the like. The drying may be carried out under reduced pressures and at various temperatures. The temperatures may range from about ambient temperature to about 100° C., for a time period that produces the desired result.

Optionally the resulted product may be converted into its acid-addition salt by reacting it with a pharmaceutically acceptable acid. Examples of such acids include: inorganic acids, such as, for example, hydrochloric acid, sulfuric acid, phosphoric acid, hydrobromic acid, and the like; and organic acids, such as, for example, oxalic acid, maleic acid, fumaric acid, malic acid, tartaric acid, citric acid, benzoic acid, and the like.

The present invention also includes processes for purifying prasugrel or its pharmaceutically acceptable salts comprising at least one of the steps of:

(a) providing a solution of prasugrel or its salt in a solvent;

(b) isolating the solid from the said solution; and

(c) optionally, drying the solid to provide pure prasugrel or its salt.

Purification Step (a) involves providing a solution of prasugrel or its salt in suitable solvents. The solution of prasugrel or its salt may be from a reaction mixture containing prasugrel or its salts obtained in the course of manufacture, or dissolution of prasugrel or its salts in a suitable solvent, or any of the processes described in the art.

Any form of prasugrel or its salts, such as, for example, anhydrous crystalline, amorphous, crystalline hydrate, or mixtures of amorphous and crystalline forms of prasugrel or its salts, in any proportions, obtained by any method, is acceptable for forming the solution.

The solution of prasugrel or its salts can be obtained by the dissolution of prasugrel or its salts in a solvent. The solvent may be chemically inert with respect to dissolved solute. Suitable volatile solvents that may be utilized for the purification of prasugrel or its salts include and are not limited to: alcoholic solvents, such as, for example, methanol, ethanol, isopropyl alcohol, and n-propanol; halogenated solvents, such as, for example, dichloromethane, 1,2-dichloroethane, chloroform, and carbon tetrachloride; ketone solvents, such as, for example, acetone, ethyl methyl ketone, and methyl isobutyl ketone; esters, such as, for example, ethyl acetate, n-propyl acetate, n-butyl acetate, isobutyl acetate, and t-butyl acetate; ether solvents, such as, for example, diethyl ether, dimethyl ether, diisopropyl ether, methyl tertiary-butyl ether, tetrahydrofuran, and 1,4-dioxane; hydrocarbon solvents, such as, for example, toluene, xylene, n-heptane, cyclohexane and n-hexane; nitrile solvents, such as, for example, acetonitrile and propionitrile;

Prasugrel or its salts may be mixed with sufficient amount of the solvent to provide solution of prasugrel or its salts at or below the reflux temperature of the solvent. Optionally, the solution obtained above can be filtered to remove the undissolved particles. The solution can be filtered by passing through paper, glass fiber, or other membrane material, or a clarifying agent, such as, for example, diatomaceous earth.

Purification Step (b) involves isolation of solid from the solution of Purification Step (a).

Suitable techniques used for isolation of solid from the solution include techniques, such as, for example, crystallization, slurrying, or trituration in a suitable solvent.

Optionally, crystallization may be enhanced by methods, such as, for example, cooling, partial removal of the solvent from the solution, seeding, adding an anti-solvent to the solution, or a combination thereof.

The obtained solution is optionally concentrated to a certain extent and cooled to suitable temperatures where the precipitation of the prasugrel or its salts begins from the solution, converting the solution into slurry.

The solution may be maintained further at a temperature lower than the concentration temperature, such as, for example, below about 40° C., for a period of time as required for a complete separation of the solid. The exact cooling temperature and time required for complete crystallization can be readily determined by a person skilled in the art and will also depend on parameters, such as, for example, concentration and temperature of the solution or slurry.

The solid can be isolated by conventional techniques, such as, for example, filtering, decanting, centrifuging and the like, or by filtering under an inert atmosphere using gases, such as, for example, nitrogen, and the like.

Purification Step (c) involves optionally drying the solid to obtain prasugrel or its salts.

Drying may be suitably carried out by any methods, such as, for example, use of a tray dryer, vacuum oven, air oven, fluidized bed drier, spin flash dryer, flash dryer, and the like. The drying may be carried out under reduced pressures and at various temperatures. The temperatures may range from about ambient temperature to about 100° C., for a time period that produces the desired result.

Optionally, the resulting prasugrel may be converted into an acid-addition salt by reacting it with a pharmaceutically acceptable acid. Examples of such acids include: inorganic acids, such as, for example, hydrochloric acid, sulfuric acid, phosphoric acid, hydrobromic acid, and the like; and organic acids, such as, for example, oxalic acid, maleic acid, fumaric acid, malic acid, tartaric acid, citric acid, benzoic acid, and the like.

Optionally, the above described steps of the invention can be adapted to form the basis of a continuous crystallization process to get substantially pure of prasugrel or its salts typically equal to or greater than about 95%, or greater than about 99%, or greater than about 99.5%, by weight as determined using HPLC.

Prasugrel of Formula I or its pharmaceutically acceptable salts obtained according to the processes of the present invention may be further purified by slurrying in a solvent. The solvents that may be utilized for this purification step are the same as the solvents that are discussed in the earlier purification step.

The present invention includes substantially pure prasugrel of Formula I or any of its pharmaceutically acceptable salts.

As used herein “substantially pure” refers to chemical purity. Prasugrel of Formula I or a pharmaceutically acceptable salt thereof comprises less than about 0.5% of total impurities, or less than about 0.1% of total impurities.

The present invention includes novel intermediates, designated as Formula III and Formula VIII, which are useful in the synthesis of prasugrel,

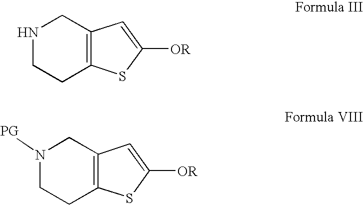

wherein R is a hydroxyl-protecting group, such as, for example, acetyl, tetrahydropyranyl, tetrahydrothiopyranyl, tetrahydrofuranyl, tetrahydrothiofuranyl, allyl, cinnamyl etc., and wherein PG is a nitrogen-protecting group, such as, for example, trityl or t-butyloxycarbonyl (Boc). For example, R is acetyl and PG is trityl.

The present invention includes processes for preparing prasugrel or its pharmaceutically acceptable salts comprising at least one of the steps of:

(a) oxidizing 5-trityl-4,5,6,7-tetrahydrothieno[3,2-c]pyridine of Formula VI,

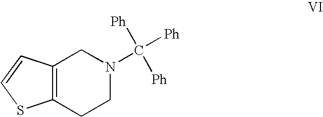

where Ph is phenyl, to provide 5-trityl-5,6,7,7a-tetrahydrothieno[3,2-c]pyridine-2(4H)-one of Formula VII;

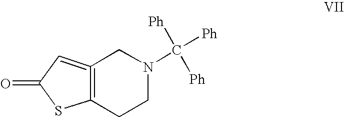

b) reacting the compound of Formula VII with an acetylating agent, such as, for example, acetic anhydride or acetyl chloride, to provide 5-trityl-4,5,6,7-tetrahydrothino[3,2-c]pyridin-2-yl acetate of Formula VIII-A;

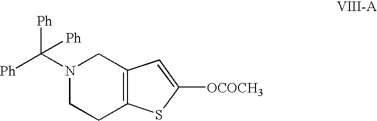

c) removing the trityl function from compound of Formula VIII-A to form the compound of Formula III-A or its acid addition salt;

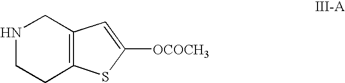

(d) reacting the compound of Formula III with the compound of Formula II

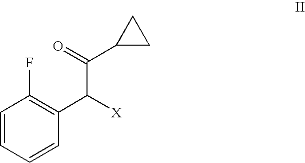

wherein X is halogen, such as, for example, bromine or chlorine; and

(e) optionally converting the obtained prasugrel into its pharmaceutically acceptable salt.

For example, the process for preparing prasugrel may be schematically represented in Scheme 5.

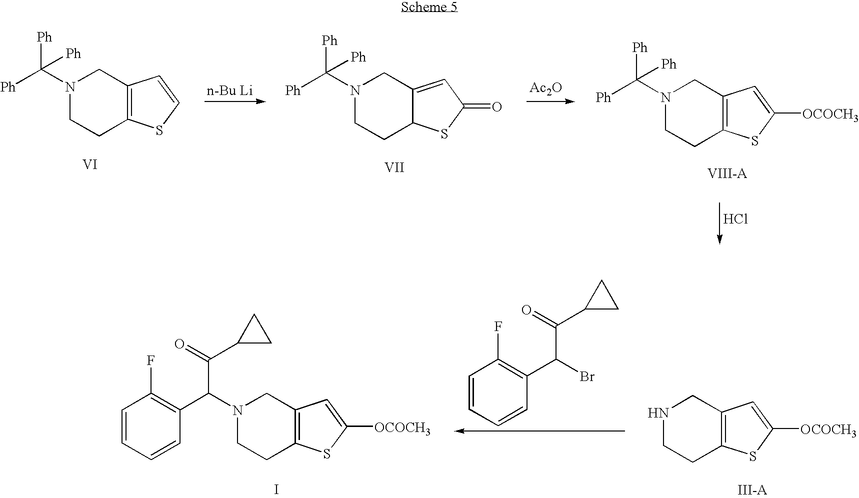

The compound of Formula VI of the present invention can be prepared by the reaction of 4,5,6,7-tetrahydrothieno[3,2-c]pyridine hydrochloride with trityl chloride in presence of organic base, such as, for example, triethylamine. The compound of Formula VI may be further purified by suitable purification techniques, such as, for example, crystallization or making a slurry, or a combination thereof, in a suitable solvent like alcohol. The compound of Formula VI may have purity greater than or equal to 95% by weight as determined using HPLC, and yield not less than 70%.

Step (a) may be carried out in presence of an oxidizing agent, such as, for example, hydrogen peroxide, and in presence of 1-3 molar equivalents of alkyl borate and 1-3 molar equivalents of alkyllithium reagent or a lithium amide. The reagents that can be used include and are not limited to, n-butyl lithium, t-butyl lithium, lithium diisopropylamide, and the like.

Alkyl borate that may be used in Step (a) can be, for example, tri-n-butyl borate, tri-t-butyl borate, trimethyl borate, trimethylene borate, triisopropyl borate, and the like.

For example, the alkyllithium reagent that may be used in step (a) can be n-butyl lithium.

Suitable temperature for conducting Step (a) may range from about −30° C. to about 50° C., or about −15° C. to about 35° C.

The solvents that may used for this step include and are not limited to hydrocarbon solvents, such as, for example, toluene, xylene, n-hexane, n-heptane, cyclohexane, and the like; ethers, such as, for example, tetrahydrofuran, 1,4-dioxane, diisopropyl ether, tetrahydrofuran, and the like; or mixtures thereof.

Water may be added after the completion of the reaction and layers may be separated. If required, a water immiscible solvent may be added before the separation of layers.

The compound may be isolated by removal of the solvent. The solvent may be removed using any suitable methods, such as, for example, evaporation, atmospheric distillation, or distillation under vacuum.

The solid may be isolated from the reaction mass by adding solvent, such as, for example, acetone, isopropyl alcohol, n-hexane, n-heptane, diisopropyl ether, etc. The reaction mass may be optionally cooled before isolation. The techniques that are described above may be used for the isolation and drying may be adopted for this step.

The compound VII obtained from this reaction may be used in the next reaction step, without isolation from the reaction mixture or in the form of a crude product. If desired, it may be isolated from the reaction mixture by ordinary methods, and it can be easily purified by means of separation, for example, recrystallization, distillation, and chromatography.

The compound of Formula VII obtained from present process may have purity greater than or equal to 90%, or greater than 95%, by weight as determined by HPLC.

Step (b) involves reaction of compound of Formula VII with acetic anhydride or acetyl chloride, optionally in the presence of a suitable base and a solvent.

The reaction may be conducted in the presence or absence of a base. Bases that are useful in the reaction include and are not limited to: inorganic bases, such as, for example, alkali metal or alkaline earth metal carbonates, hydrogen carbonates, hydroxides, oxides, hydrides, carboxylates, and alkoxides, e.g., sodium carbonate, sodium hydrogen carbonate, potassium carbonate, cesium carbonate, sodium hydroxide, potassium hydroxide, potassium carbonate, potassium t-butoxide, sodium t-butoxide, calcium oxide, sodium acetate, sodium methoxide, and the like; and organic bases, such as, for example, for example, tertiary amines, e.g., triethylamine, N,N-diisopropylethylamine, N,N-diethylethanamine, N-(1-methylethyl)-2-propanamine, 4-ethylmorpholine, 1,4-diazabicyclo[2.2.2]-octane, N-methyl morpholine, pyridine, sodium hydride, and the like or any mixtures thereof.

The solvents that may be utilized for this step include and are not limited to: alcohols, such as, for example, methanol, ethanol, isopropyl alcohol, and n-propanol; halogenated hydrocarbons, such as, for example, dichloromethane, 1,2-dichloroethane, chloroform, and carbon tetrachloride; ketones, such as, for example, acetone, ethylmethyl ketone, and methyl isobutyl ketone; ethers, such as, for example, diethyl ether, dimethyl ether, diisopropyl ether, methyl tertiary-butyl ether, 1,1′-oxybisethane, tetrahydrofuran, and 1,4-dioxane; hydrocarbons, such as, for example, n-heptane, cyclohexane and n-hexane; aromatic solvents, e.g., benzene, toluene, xylene, chlorobenzene, and methoxybenzene; nitriles, such as, for example, acetonitrile and propionitrile; dimethylsulfoxide (DMSO);
N,N-dimethylformamide (DMF); N,N-dimethylacetamide; pyridine; 1,3-dimethyl-3,4,5,6-tetrahydro-2(1H)-pyrimidinone; 1,3-dimethyl-2-imidazolidinone; 1,1,3,3-tetramethylurea; 1-methyl-2-pyrrolidinone; nitrobenzene; water; and mixtures thereof. For example, the solvent is N,N-dimethylformamide (DMF), dimethylsulfoxide (DMSO), N,N-dimethylacetamide (DMA), dichloromethane, ethylenedichloride, and chloroform; or mixtures thereof in various proportions. For example, the solvent is N,N-dimethylformamide (DMF).

The reaction may be carried out for any desired time periods to achieve the desired product yield and purity. The reaction times vary from about 30 minutes to about 10 hours, or longer.

The reaction may be conducted at temperatures ranging from about 0° C. to about 50° C.

After completion of the reaction, the obtained compound of Formula VIII-A is extracted into a suitable solvent and then the solid can be crystallized using suitable techniques.

Extraction can be done by providing the two phase system, including mixing the reaction mixture in a suitable solvent system at a room temperature until a clear solution is obtained and allowing the mixture to separate into two phases.

Suitable techniques used for isolation of solid include techniques, such as, for example, crystallization, slurrying, or trituration in a suitable solvent.

Optionally, crystallization may be enhanced by methods, such as, for example, cooling, partial removal of the solvent from the solution, seeding, adding an anti-solvent to the solution, or a combination thereof.

Suitable solvents used for extraction and isolation of solid include but are not limited to water; esters, such as, for example, ethyl acetate, n-propyl acetate, n-butyl acetate, tertiary-butyl acetate, and the like; alcohols, such as, for example, methanol, ethanol, isopropyl alcohol, n-butanol, tertiary-butyl alcohol, and the like; halogenated solvents, such as, for example, dichloromethane, ethylenedichloride, chloroform, and the like.

The techniques that are described above may be used for the isolation and drying may be adopted for this step.

The compound VIII obtained from this reaction may be used in the next reaction step, without isolation from the reaction mixture or in the form of a crude product. If desired, it may be isolated from the reaction mixture by ordinary methods, and it can be easily purified by means of separation, for example, recrystallization, distillation and chromatography.

The compound of Formula VIII-A obtained by present process may have a yield greater than or equal to 95%.

Step (c) involves removal of a trityl group from compound of Formula VIII-A with an acid to form an intermediate of Formula III-A or its salt.

This step may be carried out in an acidic medium. Examples of useful acids include hydrochloric acid, sulfuric acid, phosphoric acid, hydrobromic acid, formic acid, acetic acid, and the like. Any other methods that accomplish the removal of protecting group without affecting the product may also be used. The source of acid may be in the form of gas, aqueous solution, or solution with an organic or inorganic solvent.

Suitable solvents that can be used in step (c) including and are not limited to alcoholic solvents, such as, for example, methanol, ethanol, isopropyl alcohol, n-butanol, tertiary-butyl alcohol, and the like; hydrocarbon solvents, such as, for example, toluene, xylene, n-hexane, n-heptane, cyclohexane, and the like; ketonic solvents, such as, for example, acetone, ethyl methyl ketone, methyl isobutyl ketone, and the like; esters, such as, for example, ethyl acetate, n-propyl acetate, n-butyl acetate, tertiary-butyl acetate, and the like; nitrile solvents, such as, for example, acetonitrile, propionitrile, and the like; halogenated solvents, such as, for example, dichloromethane, ethylene dichloride, chloroform, and the like; aprotic polar solvents, such as, for example, N,N-dimethylformamide (DMF), dimethylsulfoxide (DMSO), N,N-dimethylacetamide (DMA), and the like; or mixtures thereof; or their combinations with water in various proportions.

The reaction may be conducted at temperatures ranging from about 20° C. to reflux temperature of the solvent used.

The reaction may be carried out for any desired time periods to achieve the desired product yield and purity. The reaction times vary from about 30 minutes to about 10 hours, or longer.

The product obtained after the reaction completion may be filtered and washed with a solvent preferably with the solvent used in the reaction.

The techniques that are described above may be used for the isolation and drying may be adopted for this step.

The compound of Formula III-A obtained from this reaction may be used in the next reaction step, without isolation from the reaction mixture or in the form of a crude product. If desired, it may be isolated from the reaction mixture by ordinary methods, and it can be easily purified by means of separation, for example, recrystallization, distillation and chromatography.

The compound of Formula III-A obtained by the present process can have a yield greater than or equal to 95%.

Optionally, the compound of Formula III-A may be converted into its acid-addition salt by reacting it with a pharmaceutically acceptable acid. Examples of useful acids include: inorganic acids, such as, for example, hydrochloric acid, sulfuric acid, phosphoric acid, hydrobromic acid, and the like; and organic acids, such as, for example, oxalic acid, maleic acid, fumaric acid, malic acid, tartaric acid, citric acid, benzoic acid, and the like. The conversion of the compound of Formula III-A into its salt increases the stability of the compound and hence these salts may be stored for extended times depending on their stability after their manufacture.

Step (d) and Step (e) involves the reaction of a compound of Formula II with a compound of Formula III-A and, optionally, converting the obtained prasugrel into its pharmaceutically acceptable salt. This reaction may be carried out using the methods described above.

The present invention includes processes for preparing prasugrel or its pharmaceutically acceptable salt comprising at least one of the steps of:

(a) treating 5-trityl-5,6,7,7a-tetrahydrothieno[3,2-c]pyridin-2(4H)-one of Formula VII

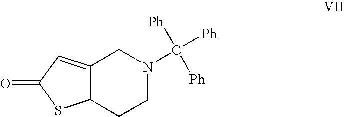

with hydrochloric acid to provide the compound of Formula V or its salt

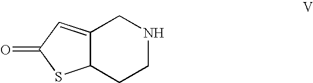

(b) condensing 2-fluoro-α-cyclopropylcarbonyl bromide of Formula II,

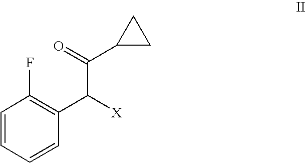

wherein X is bromide, with a compound of Formula V or its salt or tautomer thereof in presence of a base to form 5-[2-cyclopropyl-1-(2-fluorophenyl)-2-oxoethyl]-5,6,7,7a-tetrahydrothieno[3,2-c]pyridin-2(4H)-one of Formula IX;

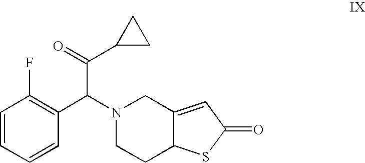

(c) reacting the compound of Formula IX with an acetylating agent, such as, for example, acetic anhydride or acetyl chloride, optionally in the presence of a base and/or solvent to afford prasugrel; and

(d) optionally, converting obtained prasugrel into its pharmaceutically acceptable salt.

For example, the process for preparing prasugrel may be schematically represented in Scheme 6.

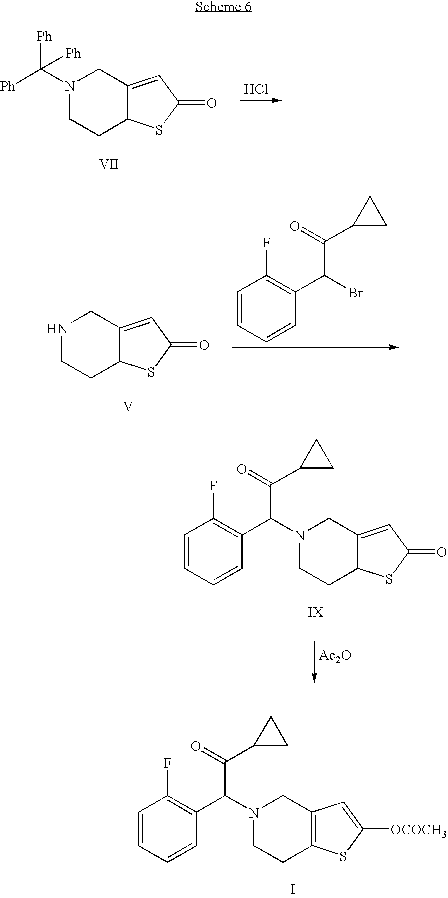

Step (a) involves treating 5-trityl-5,6,7,7a-tetrahydrothieno[3,2-c]pyridin-2(4H)-one of Formula VII with hydrochloric acid to provide the compound of Formula V or its salt.

Suitable solvents that may be used to prepare the compound of the Formula V include and are not limited to, alcohols, such as, for example, methanol, ethanol, isopropyl alcohol and the like; ketonic solvents, such as, for example, acetone, ethyl methyl ketone, and methyl isobutyl ketone; or mixtures thereof. The source of hydrochloric acid that may be used in the form of gas, or solution in a solvent.

Suitable temperatures can range from about 20° C. to about the reflux temperature of the solvent.

For example, the concentration of hydrochloric acid in acetone for step (a) is between 2-5% at a temperature between about ambient temperature and the reflux temperature of the reaction medium to provide selective cleavage of the trityl group without affecting the thienopyridyl ring.

The compound of Formula V or its salt may be further purified using suitable purification techniques, such as, for example, crystallization, making a slurry, or a combination thereof in a suitable solvent or it can be used directly in next processing step. The compound of Formula V or its salt obtained can have purity greater than or equal to 95%.

Step (b) involves condensing pure 2-fluoro-α-cyclopropylcarbonyl bromide of Formula II, wherein the X is bromide, with the compound of Formula V or its salt in the presence of a base.

The reaction may be conducted in the presence or absence of a base. Bases that are useful in the reaction including and are not limited to: inorganic bases, such as, for example, alkali metal or alkaline earth metal carbonates, hydrogen carbonates, hydroxides, oxides, carboxylates, and alkoxides, e.g., sodium carbonate, sodium hydrogen carbonate, potassium carbonate, cesium carbonate, sodium hydroxide, potassium hydroxide, potassium carbonate, potassium t-butoxide, sodium t-butoxide, calcium oxide, sodium acetate, sodium methoxide, and the like; and organic bases, such as, for example, for example, tertiary amines, e.g., triethylamine, N,N-diisopropylethylamine, N,N-diethylethanamine, N-(1-methylethyl)-2-propanamine, 4-ethylmorpholine, 1,4-diazabicyclo[2.2.2]-octane, N-methylmorpholine, pyridine, and the like or mixtures thereof.

The solvents that may be utilized for this step including and are not limited to: alcohols, such as, for example, methanol, ethanol, isopropyl alcohol and n-propanol; halogenated hydrocarbons, such as, for example, dichloromethane, 1,2-dichloroethane, chloroform, and carbon tetrachloride; ketones, such as, for example, acetone, ethyl methyl ketone and methyl isobutyl ketone; ethers, such as, for example, diethyl ether, dimethyl ether, diisopropyl ether, methyl tertiary-butyl ether, 1,1′-oxybisethane, tetrahydrofuran and 1,4-dioxane; hydrocarbons, such as, for example, n-heptane, cyclohexane and n-hexane; aromatic solvents, e.g. benzene, toluene, xylene, chlorobenzene, and methoxybenzene; nitriles, such as, for example, acetonitrile and propionitrile; dimethylsulfoxide (DMSO), N,N-dimethylformamide (DMF) and N,N-dimethylacetamide; pyridine; 1,3-dimethyl-3,4,5,6-tetrahydro-2(1H)-pyrimidinone; 1,3-dimethyl-2-imidazolidinone; 1,1,3,3-tetramethylurea; 1-methyl-2-pyrrolidinone; nitrobenzene; water; and mixtures thereof. For example, solvents are N,N-dimethylformamide (DMF), dimethylsulfoxide (DMSO), N,N-dimethylacetamide (DMA), dichloromethane, ethylenedichloride, and chloroform; or mixtures thereof in various proportions.

The reaction may be carried out for any desired time periods to achieve the desired product yield and purity. Typical reaction times can vary from about 1 hour to 20 hours, or longer. Suitable temperatures for conducting the reaction may range from about 0° C. to about 100° C., or about 20° C. to about 35° C.

Additionally, it may be advantageous to conduct this step under an inert atmosphere, such as, for example, for example, oxygen-free argon or nitrogen gas.

The base is used in an amount approximately 1 to 10 moles, or approximately 1 to 5 moles, per mole of the compound of Formula II.

After completion of the reaction, compound of Formula IX may be extracted into a solvent. Extraction may be done by providing a two phase system including mixing the reaction mixture in suitable solvent system at room temperature until a clear solution is obtained and allowing the mixture to separate into two phases.

The organic layer comprising compound of Formula IX may be used directly in the next processing step or solid may be isolated.

Suitable techniques used for isolation of solid include techniques of crystallization, slurrying, or trituration in a suitable solvent.

Suitable solvents that can be used in extraction and solid isolation include but are not limited to: hydrocarbons, such as, for example, n-hexane, cyclohexane, heptane and the like; aromatic solvents, such as, for example, benzene, toluene and the like; alcohol solvents, such as, for example, methanol, ethanol, isopropanol, and the like; esters, such as, for example, ethyl acetate, methyl acetate, isopropyl acetate and the like; and halogenated solvents, such as, for example, dichloromethane or their combinations with water in various proportions.

The compound of Formula IX obtained by the present invention has purity greater than or equal to 70% by weight, as determined using HPLC.

Step (c) involves reaction of compound of Formula IX obtained from Step (b) with acetic anhydride or acetyl chloride in presence of a base to provide prasugrel.

The bases that can be used in Step (c) include and are not limited to, sodium hydride, sodium methoxide, sodium amide, potassium carbonate, sodium carbonate, cesium carbonate, potassium t-butoxide, sodium t-butoxide, potassium hydroxide, sodium hydroxide, potassium bicarbonate, triethylamine, N,N-diisopropylethylamine or mixtures thereof.

The quantity of the base that can be used in the Step (c) may range from about 0.5 to about 3 moles, per mole of the compound of Formula IV.

Suitable solvents that may be used in the Step (c) include but are not limited to aprotic polar solvents, such as, for example, N,N-dimethylformamide (DMF), dimethylsulfoxide (DMSO), N,N-dimethylacetamide (DMA) and the like; halogenated solvents, such as, for example, dichloromethane, ethylene dichloride, chloroform and the like; or mixtures thereof in various proportions.

Suitable temperatures range from about −30° C. to about 50° C.

After completion of the reaction, the product, prasugrel, may be extracted into a suitable solvent.

Suitable solvents which may be used for extraction of prasugrel include and are not limited to, chlorinated solvents, such as, for example, dichloromethane, dichloroethane, chloroform, and the like; esters, such as, for example, ethyl acetate, methyl acetate, isopropyl acetate and the like.

The organic layer that includes prasugrel may be used directly in the next processing step or it may be crystallized in a suitable solvent.

Prasugrel obtained by the processes described herein may be converted to a pharmaceutically acceptable salt.

Pharmaceutically acceptable salts include acid addition salts formed with inorganic acids, such as, for example, hydrochloric acid, hydrobromic acid, hydroiodic acid, sulfuric acid, nitric acid, phosphoric acid, and the like; or with organic acids, such as, for example, acetic acid, propionic acid, hexanoic acid, heptanoic acid, malonic acid, succinic acid, malic acid, tartaric acid, citric acid, oxalic acid, and the like.

The present invention includes processes for preparing an intermediate, 2-fluoro-α-cyclopropylcarbonyl bromide of Formula II-A,

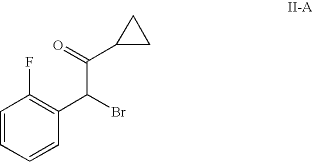

which is useful in the preparation of prasugrel, which processes comprise at least one of the steps of:

(a) reacting 2-fluorobenzylbromide with cyclopropyl cyanide to provide cyclopropyl 2-fluorobenzyl ketone of Formula X

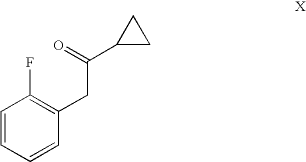

(b) brominating cyclopropyl-2-fluorobenzyl ketone of Formula X with N-bromosuccinamide (NBS) in presence of azobis(isobutyronitrile) to provide compound of Formula II.

Step (a) involves reaction of 2-fluorobenzylbromide with cyclopropyl cyanide followed by purification to provide cyclopropyl 2-fluorobenzyl ketone of Formula X.

2-fluorobenzylbromide may be treated with cyclopropyl cyanide in the presence of magnesium and a suitable solvent, such as, for example, ether solvents, which include tetrahydrofuran, 1,4-dioxane, diisopropyl ether, and the like, or mixtures thereof.

Suitable temperatures for conducting the reaction may range from about 0° C. to about 50° C., or about 25° C. to about 35° C.

The obtained compound of Formula X may be further purified using suitable purification techniques, such as, for example, crystallization, making a slurry, extractions into a suitable solvent, fractional distillation techniques, or a combination thereof, to provide purity greater than 80% by weight as determined using HPLC.

Step (b) involves bromination of cyclopropyl-2-fluorobenzyl ketone of Formula X with N-bromosuccinamide (NBS) in the presence of azobis(isobutyronitrile) to provide pure 2-fluoro-α-cyclopropylcarbonyl bromide of Formula II.

The free radical initiator azobis(isobutyronitrile) or “AIBN” may be used as a catalyst in the process of the present invention. AIBN is an efficient source of radicals and is used in the production of bromine radicals. AIBN is safe to use in commercial production because no oxygenated residues are produced.

A free radical initiator, such as, for example, AIBN, can be used in ratio of about 0.01 to about 0.2 moles per mole of the compound of Formula V.

The bromination reaction may be carried out in an acid medium. The acids may include p-toluenesulfonic acid, and the like.

The bromination reaction can be performed in the presence of suitable solvents that include and are not limited to halogenated solvents, such as, for example, carbon tetrachloride, chloroform, methylene dichloride, or mixtures thereof, or their combinations with water in various proportions. For example, methylene dichloride is used as a solvent for bromination.

Suitable temperatures for conducting the bromination reaction may range from about −30° C. to about 100° C., or about 40° C. to about 50° C.

2-fluoro-α-cyclopropylcarbonyl bromide obtained by the process of the present invention may be purified by recrystallization or making a slurry, or a combination thereof, in a suitable solvent, if desired.

Suitable solvents which can be used for crystallization or to make a slurry include but are not limited to: hydrocarbons, such as, for example, n-hexane, cyclohexane, heptane, and the like; aromatic solvents, such as, for example, benzene, toluene, and the like; alcohol solvents, such as, for example, methanol, ethanol, isopropanol, and the like; esters, such as, for example, ethyl acetate, methyl acetate, isopropyl acetate, and the like; and halogenated solvents, such as, for example, dichloromethane; or combinations thereof with water in various proportions.

2-fluoro-α-cyclopropylcarbonyl bromide obtained by the present process may have a purity greater than or equal to 70% as determined by HPLC and a yield greater than 75%.

An amorphous form of prasugrel or its salts can be prepared using suitable techniques, such as, for example, distillation, spray drying, freeze drying (lyophilization), agitated thin film drying (“ATFD”), ball milling, grinding, and the like.

The reaction conditions applied in the processes of the present invention are environmentally friendly, efficient, and mild to the product, leading to a much higher yield, purity, and adaptability to an industrial scale.

Purification techniques, such as, for example, crystallization, instead of column chromatography, have the advantage of reducing the volumes of solvent required, which also facilitate good purity and yield.

New solid forms of pharmaceutically useful compounds provide an opportunity to improve the characteristics of these products, such as, for example, stability, solubility, and formulation processability. Although the existence, let alone the properties, of solid forms (e.g., crystalline polymorphs, amorphous, etc.) for any given chemical compound cannot be predicted, active pharmaceutical ingredients, like prasugrel hydrochloride, may give rise to a variety of solid forms having different physical characteristics and distinct physicochemical properties that may be characterized by various analytical methods, e.g., XRD patterns, IR absorption spectra, solid state NMR spectra and DSC thermograms, TGA curves, etc. In some cases, different forms of the same drug can exhibit different solubility properties and, therefore, different dissolution rates. This variation may result in finished dosage forms with different bioavailability between various production lots of formulated pharmaceutical products. Since polymorphic forms may vary in their physical and chemical properties, regulatory authorities require identification of the polymorphic nature of the active pharmaceutical ingredients so that there is no variation in the bioavailability of the finished dosage forms.

The present invention includes polymorphic forms of prasugrel hydrochloride and processes for their preparation.

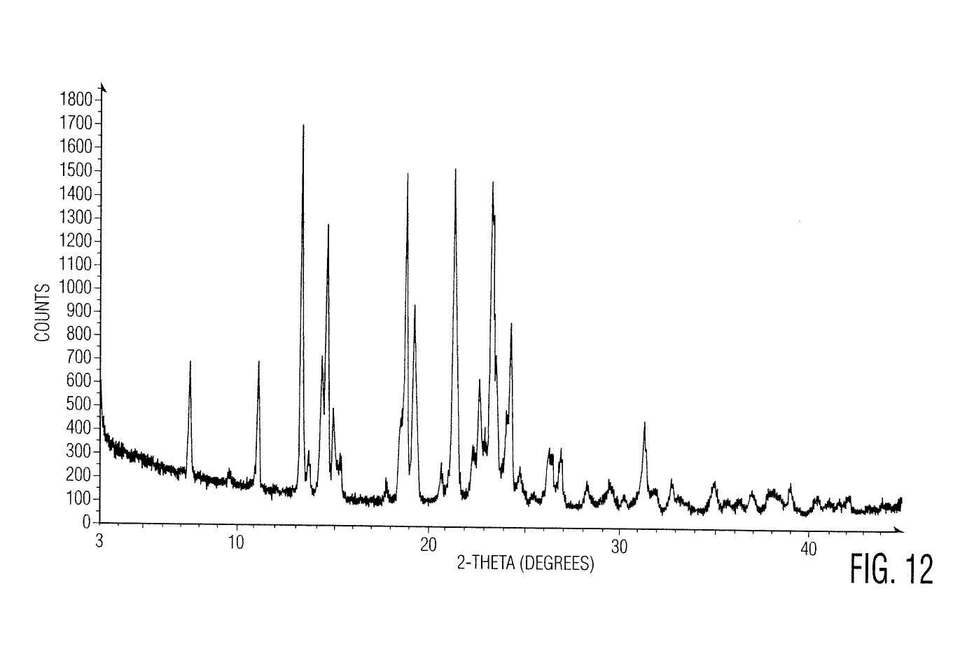

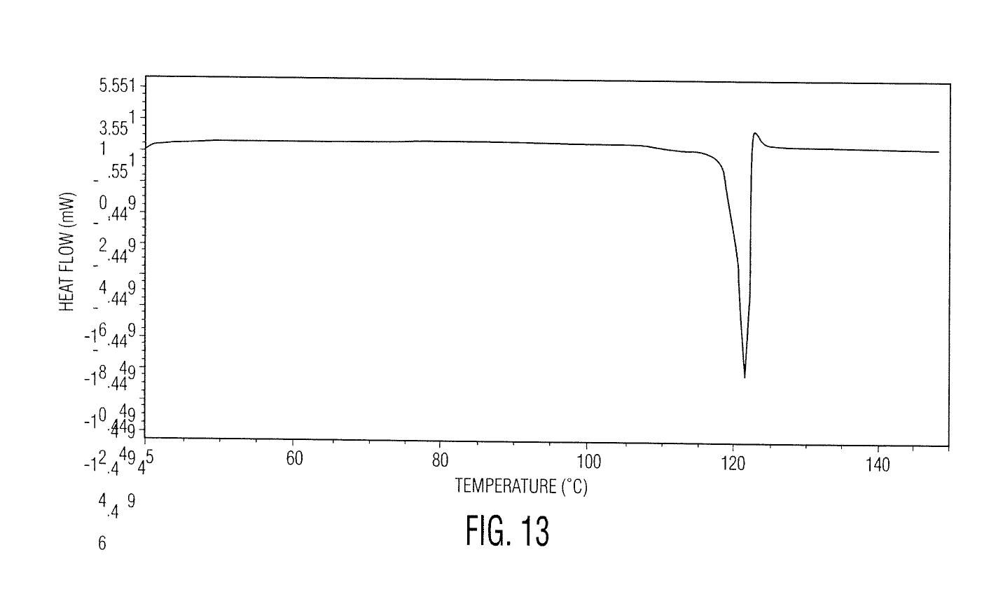

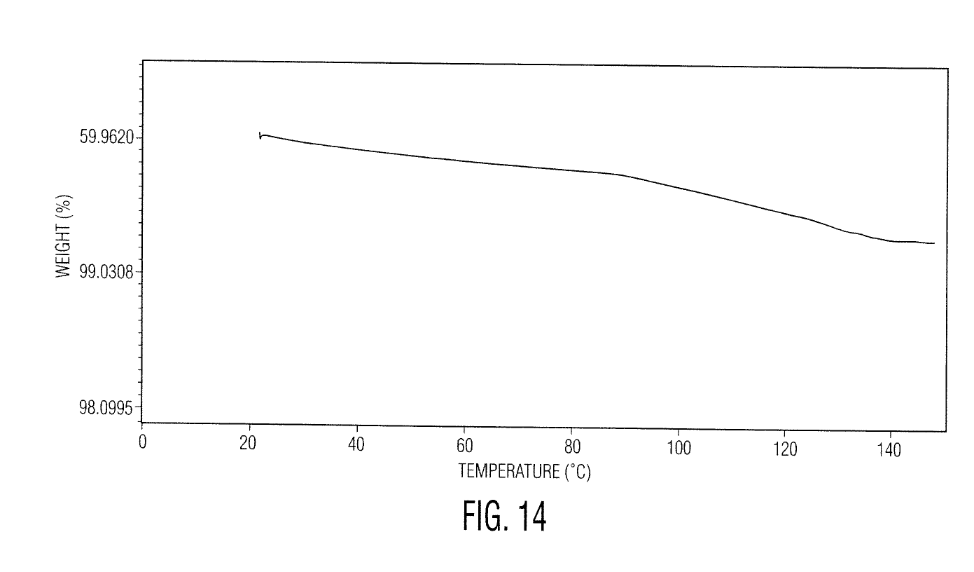

For example, there is provided a crystalline free base form of prasugrel, which may be characterized by an X-ray powder diffraction pattern with peaks at diffraction angles 2-theta of about 7.6, 11, 13.3, 14.3, 14.6, 14.9, 18.4, 18.7, 19.1, 21.3, 22.5, 23.2, 23.4, 23.9, 24.2, and 31.2, ±0.2 degrees. An example of an XRD pattern for this crystalline form is shown in FIG. 12. The crystalline form is further characterized by having a DSC thermogram with an endotherm peak at about 121° C. as substantially represented in FIG. 13 and a TGA curve corresponding to a weight loss of about 0.6%, as substantially shown in FIG. 14.

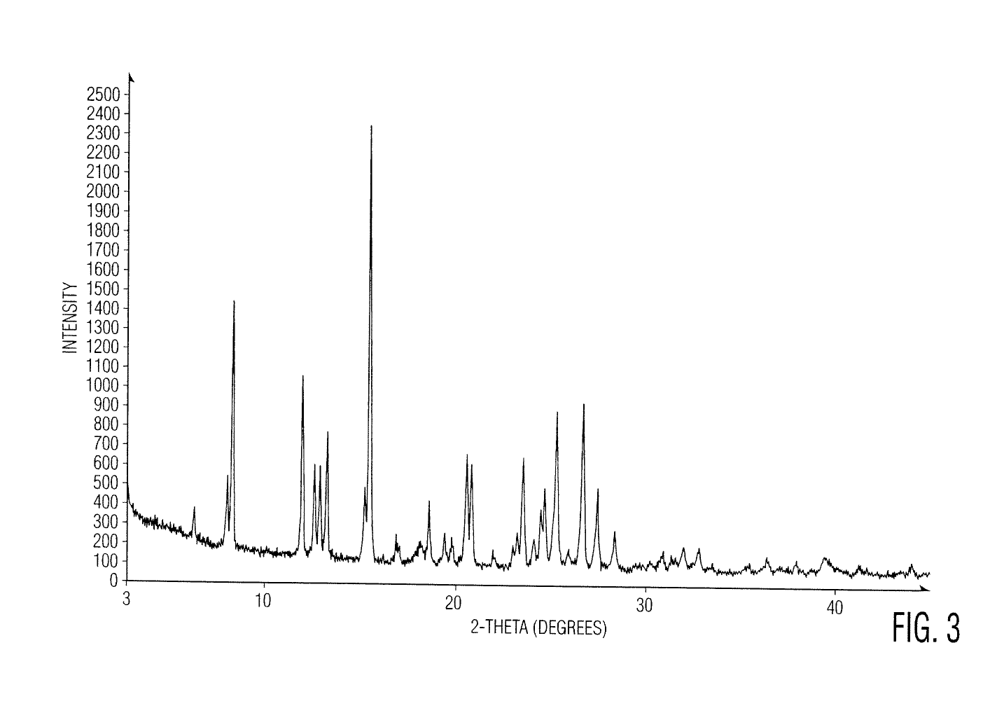

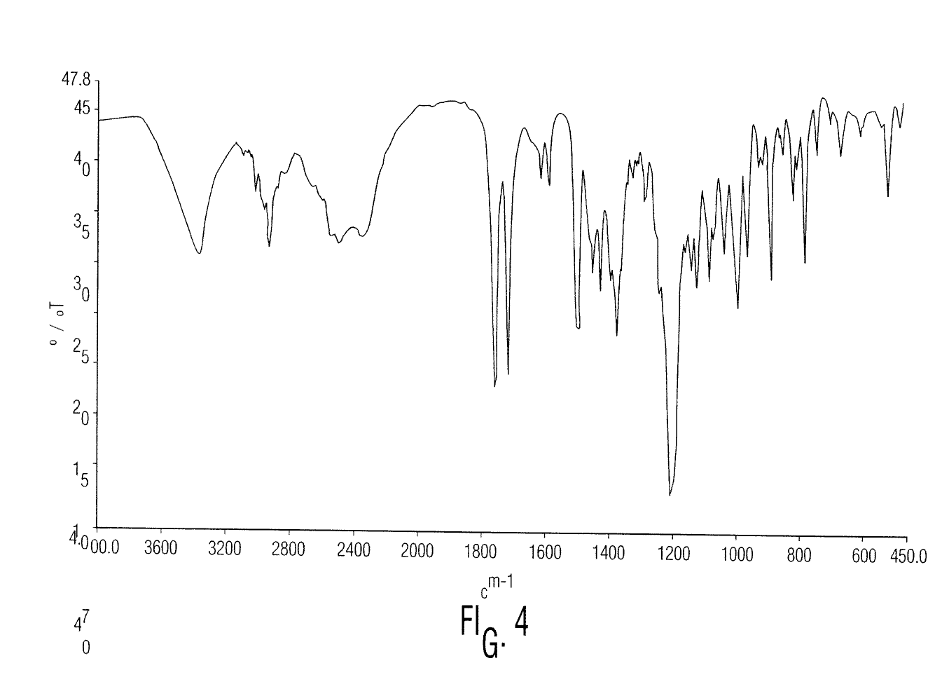

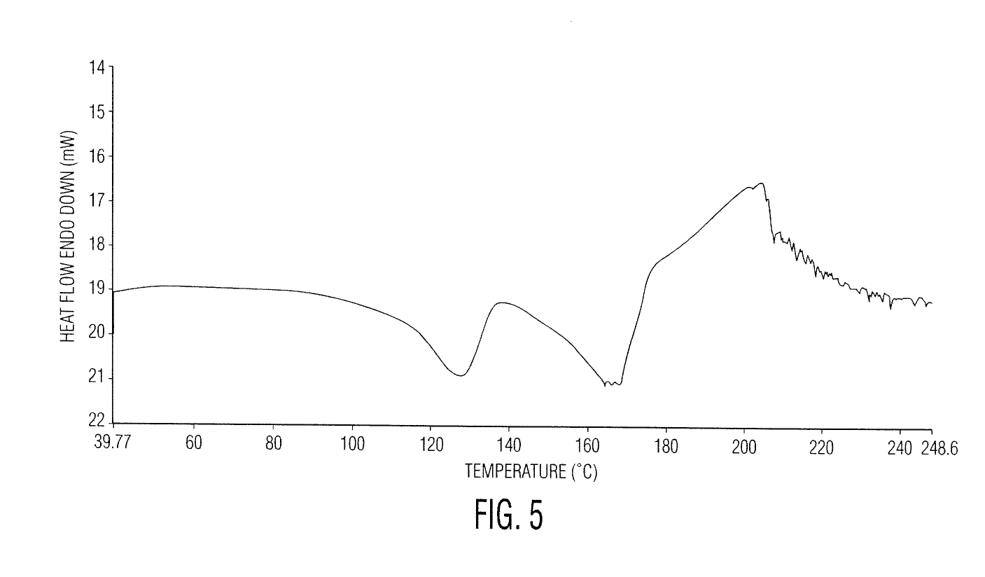

For example, there is provided a crystalline Form C of prasugrel hydrochloride. Form C may be characterized by a DSC thermogram as substantially represented in FIG. 5 with endotherm onset at about 111° C. Form C may also be characterized by TGA curve corresponding to a weight loss of about 11%. Form C may also be characterized by an IR spectrum. An example of such IR spectra, in a KBr pellet, is represented in FIG. 4. Form C may also be characterized by an XRD pattern with peaks at diffraction angles 2-theta of about 8, 8.4, 11.9, 12.5, 12.8, 13.2, 15.2, 15.4, 18.5, 20.4, 20.7, 23.4, 24.4, 24.6, 25.2, 25.8, 26.6, and 27.3, ±0.2 degrees. An example of an XRD pattern for Form C is shown in FIG. 3.

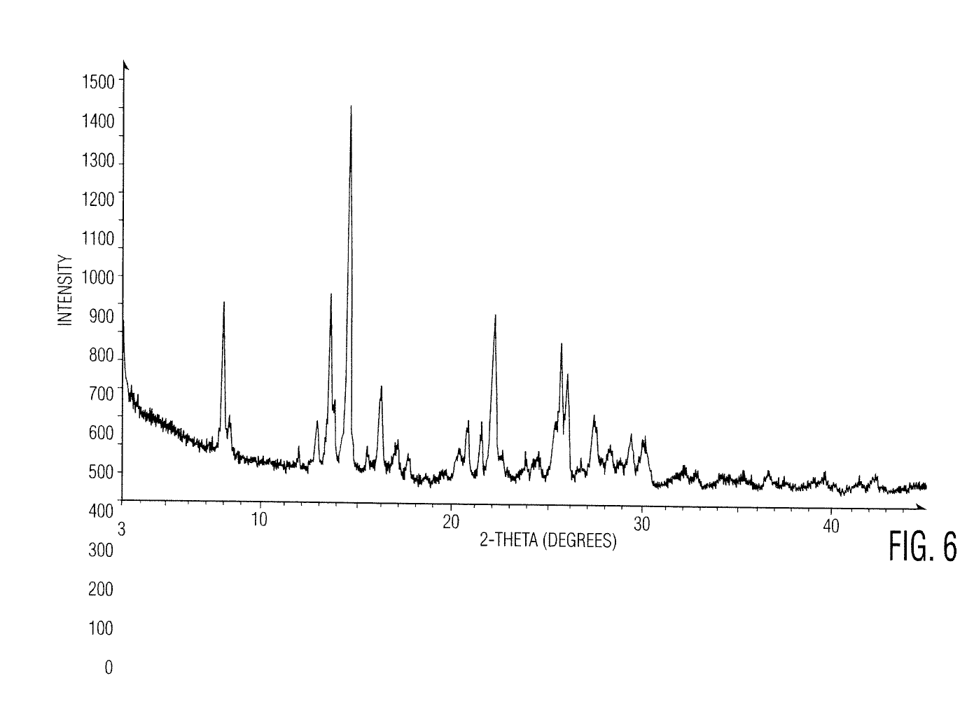

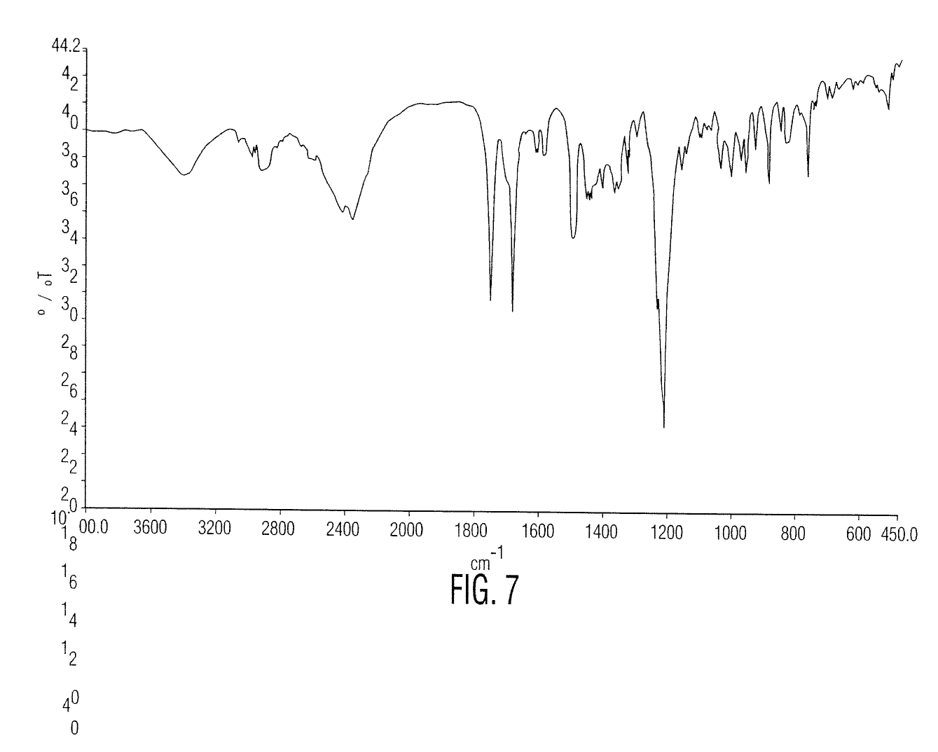

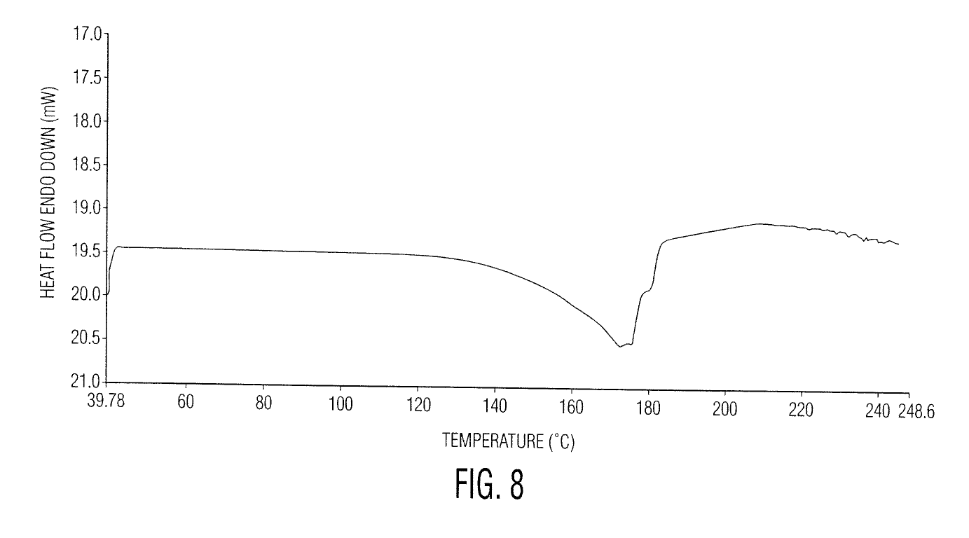

For example, there is provided a crystalline Form D of prasugrel hydrochloride. Form D may be characterized by DSC thermogram, an example of which is shown in FIG. 8, with endotherm onset at about 155° C. Form D may be characterized by an XRD pattern with peaks at diffraction angles 2-theta of about 8.1, 13.6, 14.6, 16.2, 20.7, 22.1, 24.5, 25.5, 25.9, 27.4, 30.1, and 32.8, ±0.2 degrees. An example of an XRD pattern is shown in FIG. 6. Form D may be also characterized by an IR spectrum in a KBr pellet as represented by an example shown in FIG. 7.

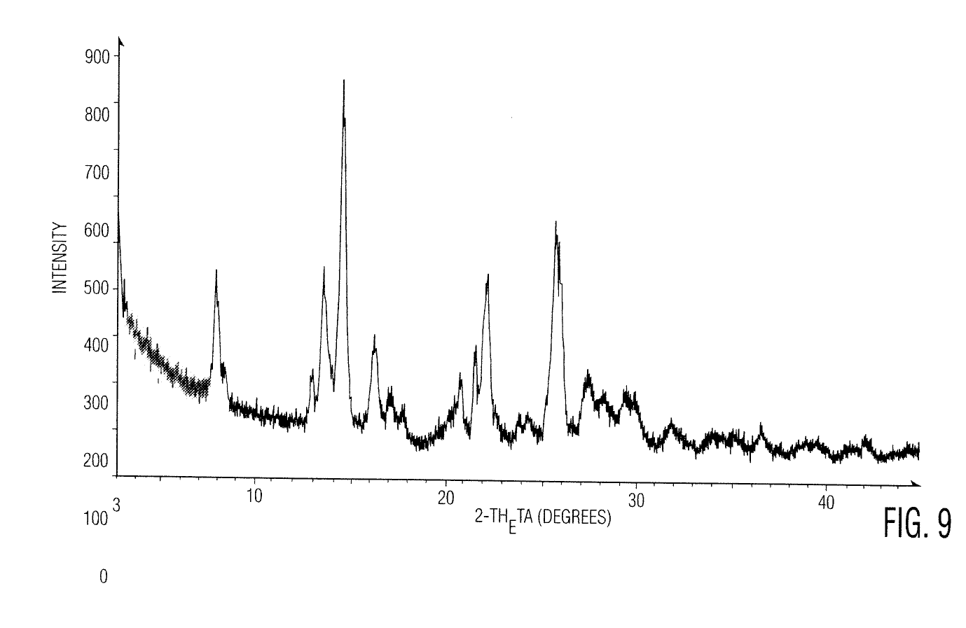

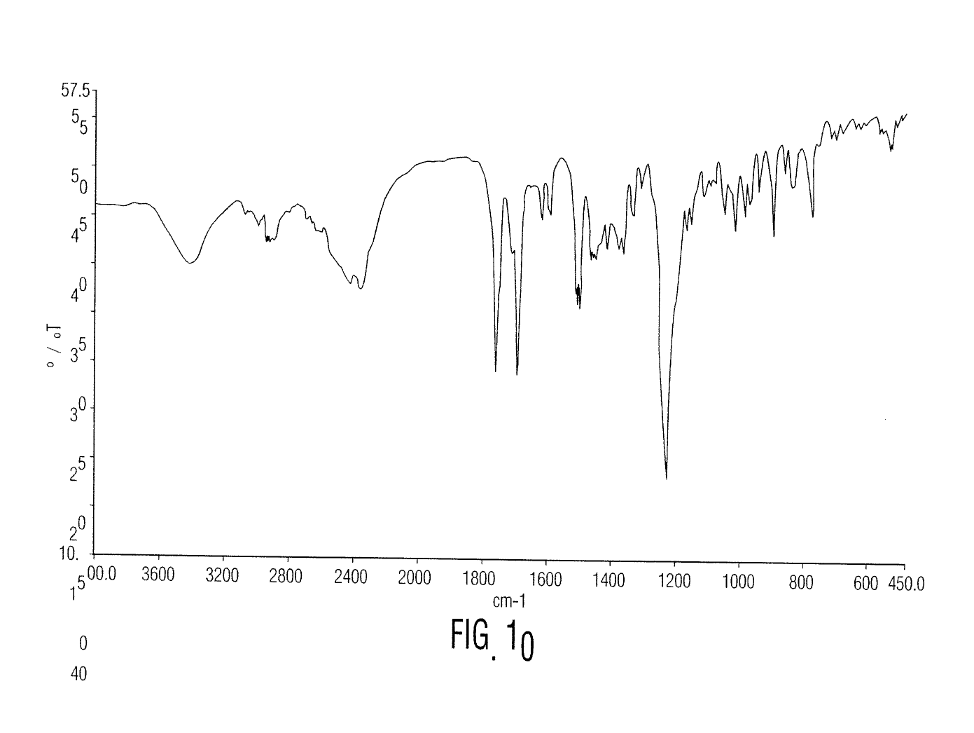

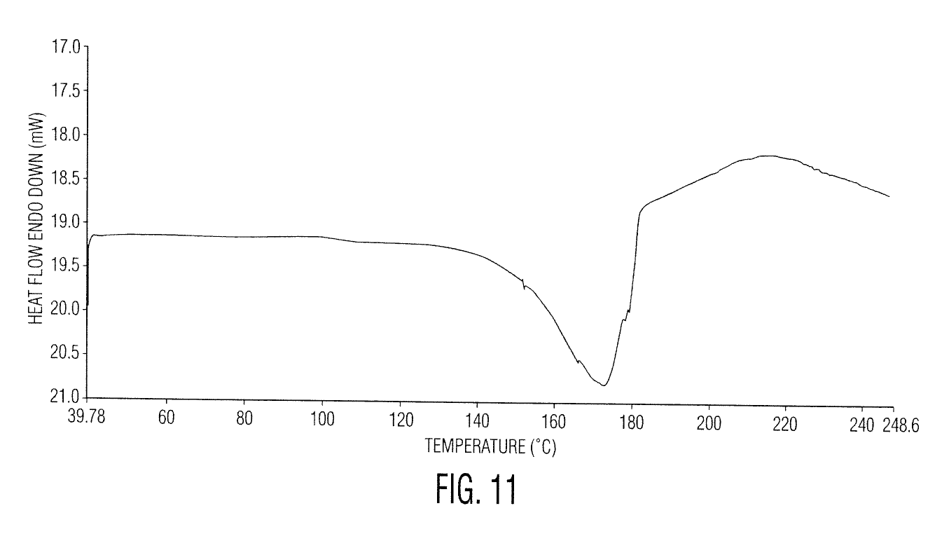

For example, there is provided a crystalline Form E, which may be characterized by DSC having thermogram with endotherm peak at about 151° C. An example of DSC thermogram is shown in FIG. 11. Form E may be characterized by XRD pattern with peaks at diffraction angles 2-theta of about 8, 8.4, 12.9, 13.5, 14.5, 16.1, 20.6, 21.4, 22, 25.6, 25.8, and 27.3, ±0.2 degrees. An example of an XRD pattern is shown in FIG. 9. Form E may be characterized by an IR spectrum. An example of an IR spectrum for Form E in a KBr pellet is shown in FIG. 10.

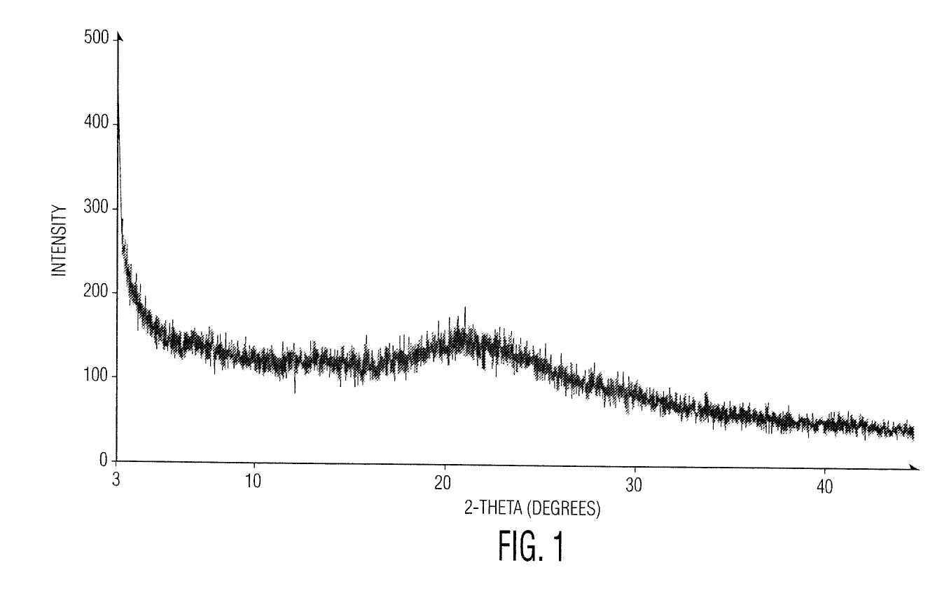

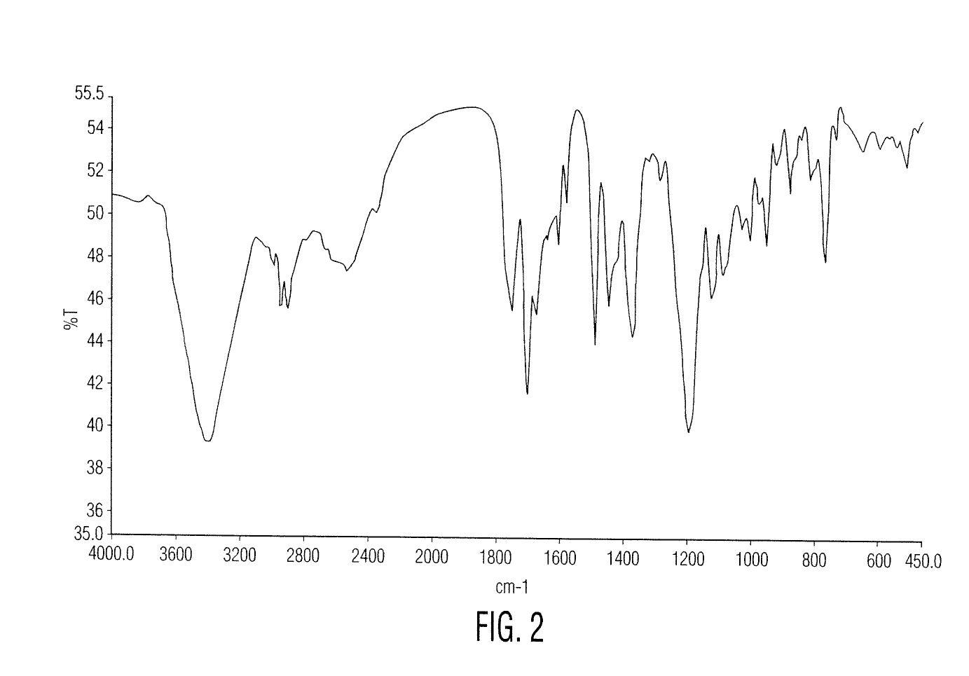

For example, there is provided an amorphous form of prasugrel hydrochloride. An example of an XRD pattern for amorphous prasugrel is shown in FIG. 1. The amorphous form may be characterized by an IR spectrum. An example of an IR spectrum for the amorphous form in a KBr pellet is shown in FIG. 2.

The present invention includes processes for the preparation of prasugrel hydrochloride crystalline Form C comprising at least one of the steps of:

(a) providing a solution of prasugrel free base in 2-butanol;

(b) adding a source of hydrochloride;

(c) collecting the precipitated solid; and

(d) drying the solid obtained in step (c).

Step (a) involves preparing a solution of prasugrel free base in 2-butanol.

The solution of prasugrel free base can be prepared by the dissolution or making slurry of prasugrel free base in 2-butanol or it may be obtained from the reaction mass of previous step. Any form of prasugrel, such as, for example, anhydrous crystalline, amorphous, or mixtures of amorphous and crystalline forms of prasugrel, in any proportions, obtained by any method, is acceptable for forming the solution.

The solution of prasugrel with 2-butanol may be prepared at room temperature to reflux temperature of the solvent, depending on the quantity of solvent used.

Step (b) involves adding a source of hydrogen chloride.

Suitable sources of hydrogen chloride include and are not limited to, aqueous hydrogen chloride (5%-36%) or a solution of hydrogen chloride in an organic solvent, such as, for example, methanol, ethanol, ethyl acetate, isopropyl alcohol, acetone, or dry hydrogen chloride gas, or mixtures thereof. For example, a source of hydrogen chloride is aqueous hydrochloric acid.

Suitable temperatures for adding source of hydrogen chloride can be about −30° C. to about 60° C.

Step (c) involves collecting the precipitated solid.

The method by which the solid material is collected from the final mixture, with or without cooling below the operating temperature, can be any of techniques, such as, for example, filtration by gravity, or by suction, centrifugation, and the like. The crystals so isolated can carry a small proportion of occluded mother liquor. If desired, the crystals can be washed with a suitable solvent or mixture of solvents in various proportions to wash out the mother liquor.

Step (d) drying the product obtained in step (c).

Drying can be suitably carried out in a tray dryer, vacuum oven, air oven, fluidized bed drier, spin flash dryer, flash dryer and the like. The drying may be carried out at temperatures of about 35° C. to about 70° C. The drying can be carried out for any time periods necessary for obtaining a desired purity, such as, for example, from about 1 to about 25 hours, or longer.

The present invention includes processes for the preparation of prasugrel hydrochloride crystalline Form D comprising at least one of the steps of:

(a) providing a solution of prasugrel free base in isopropyl alcohol;

(b) adding a source of hydrochloride;

(c) collecting the precipitated solid; and

(d) drying the solid obtained in step (c).

Step (a) involves preparing a solution of prasugrel free base in isopropyl alcohol.

The solution of prasugrel free base can be prepared by the dissolution or making slurry of prasugrel free base in isopropyl alcohol or it may be obtained from the reaction mass of previous step. Any form of prasugrel, such as, for example, anhydrous crystalline, amorphous, or mixtures of amorphous and crystalline forms of prasugrel, in any proportions, obtained by any method, is acceptable for forming the solution.

The solution of prasugrel with isopropyl alcohol may be prepared at room temperature to reflux temperature of the solvent depending on the quantity of solvent used.

Step (b) involves adding a source of hydrogen chloride.

Suitable sources of hydrogen chloride include and are not limited to aqueous hydrogen chloride (5%-36%) or a solution of hydrogen chloride in an organic solvent, such as, for example, methanol, ethanol, ethyl acetate, isopropyl alcohol, acetone, or dry hydrogen chloride gas, or mixtures thereof. For example, the source of hydrogen chloride is hydrogen chloride in isopropyl alcohol.

Suitable temperatures for adding source of hydrogen chloride can be about −30° C. to about 60° C.

Step (c) involves collecting the precipitated solid.

The method by which the solid material is collected from the final mixture, with or without cooling below the operating temperature, can be any of techniques, such as, for example, filtration by gravity, or by suction, centrifugation, and the like. The crystals so isolated will carry a small proportion of occluded mother liquor. If desired, the crystals can be washed with a suitable solvent or mixture of solvents in various proportions to wash out the mother liquor.

Step (d) involves drying the product obtained in Step (c).

Drying can be suitably carried out in a tray dryer, vacuum oven, air oven, fluidized bed drier, spin flash dryer, flash dryer and the like. The drying may be carried out at temperatures of about 35° C. to about 70° C. The drying can be carried out for any time periods necessary for obtaining a desired purity, such as, for example, from about 1 to about 25 hours, or longer.

The present invention includes processes for the preparation of prasugrel hydrochloride crystalline Form E comprising at least one of the steps of:

(a) providing a solution of prasugrel free base in ethyl acetate;

(b) adding a source of hydrochloride;

(c) collecting the precipitated solid; and

(d) drying the solid obtained in step (c).

Step (a) involves preparing a solution of prasugrel free base ethyl acetate.

The solution of prasugrel free base can be prepared by the dissolution or making a slurry of prasugrel free base in ethyl acetate or it may be obtained from the reaction mass of previous step. Any form of prasugrel, such as, for example, anhydrous crystalline, amorphous, or mixtures of amorphous and crystalline forms of prasugrel, in any proportions, obtained by any method, is acceptable for forming the solution.

The solution of prasugrel with ethyl acetate may be prepared at room temperature to reflux temperature of the solvent depending on the quantity of solvent used.

Step (b) involves adding a source of hydrogen chloride.

Suitable sources of hydrogen chloride include and are not limited to, aqueous hydrogen chloride (5%-36%) or a solution of hydrogen chloride in an organic solvent, such as, for example, methanol, ethanol, ethyl acetate, isopropyl alcohol, acetone, or dry hydrogen chloride gas, or mixtures thereof. For example, the source of hydrogen chloride is hydrogen chloride in ethyl acetate.

Suitable temperatures for adding source of hydrogen chloride can be about −30° C. to about 60° C.

Step (c) collecting the precipitated solid

The method by which the solid material is collected from the final mixture, with or without cooling below the operating temperature, can be any of techniques, such as, for example, filtration by gravity, or by suction, centrifugation, and the like. The crystals so isolated will carry a small proportion of occluded mother liquor. If desired, the crystals can be washed with a suitable solvent or mixture of solvents in various proportions to wash out the mother liquor.

Step (d) drying the product obtained in Step (c).

Drying can be suitably carried out in a tray dryer, vacuum oven, air oven, fluidized bed drier, spin flash dryer, flash dryer and the like. The drying may be carried out at temperatures of about 35° C. to about 70° C. The drying can be carried out for any time periods necessary for obtaining a desired purity, such as, for example, from about 1 to about 25 hours, or longer.

The present invention includes processes for the preparation of an amorphous form of prasugrel hydrochloride comprising at least one of the steps of:

(a) providing a solution of prasugrel free base in a solvent;

(b) adding a source of hydrochloride;

(c) removing the solvent from the solution;

(d) optionally adding isopropyl alcohol to the reaction mass obtained in Step (c);

(e) optionally removing the solvent from the solution to provide the desired amorphous prasugrel hydrochloride; and

(f) drying the product obtained in Step (e).

Step (a) involves providing a solution of prasugrel free base in a solvent.

The solution of prasugrel may be provided by the dissolution or making slurry of prasugrel free base in a solvent or it may be obtained from the reaction mass of a synthesis step. Any form of prasugrel, such as, for example, anhydrous crystalline, amorphous, or mixture of crystalline and amorphous forms of prasugrel, in any proportions, obtained by any method, is acceptable for forming the solution.

The solution of prasugrel may be prepared at room temperature to reflux temperature of the solvent depending on the quantity of solvent used.

The solvent that may be used for providing solution may be from the various classes of solvents, such as, for example, alcoholic solvents, ketones, esters, ethers, halogenated solvents, hydrocarbons, nitriles, water aprotic polar solvents or mixtures thereof. These include: alcohol solvents, such as, for example, methanol, ethanol, denatured spirits, n-propanol, isopropanol, n-butanol, isobutanol, and t-butanol and the like; ketonic solvents, such as, for example, acetone, propanone, 2-butanone and the like; halogenated solvents, such as, for example, dichloromethane, 1,2-dichloroethane, chloroform, and the like; ester solvents, such as, for example, ethyl acetate, n-propyl acetate, isopropyl acetate and n-butyl acetate and the like; ether solvents, such as, for example, dimethyl ether, diethyl ether, methyl tertiary-butyl ether, ethyl methyl ether, diisopropyl ether, tetrahydrofuran, dioxane and the like. The hydrocarbon may be any solvent from this class, such as, for example, toluene, xylene, cyclohexane, n-hexane, n-heptane and the like. The nitrile solvents may include acetonitrile, propionitrile and the like, or mixtures thereof or their aqueous combinations in various ratios without limitation.

The undissolved particles may be removed suitably by filtration, centrifugation, decantation, and other techniques. The solution may be filtered by passing through paper, glass fiber, or other membrane material, or a clarifying agent, such as, for example, celite. Depending upon the equipment used and the concentration and temperature of the solution, the filtration apparatus may need to be preheated to avoid premature crystallization.

Step (b) involves adding a source of hydrochloride.

Suitable sources of hydrogen chloride including and are not limited to, aqueous hydrogen chloride (5%-36%); a solution of hydrogen chloride in an organic solvent, such as, for example, methanol, ethanol, ethyl acetate, isopropyl alcohol, acetone; dry hydrogen chloride gas; or mixtures thereof.

Suitable temperatures for adding source of hydrogen chloride can be about −30° C. to about 60° C.

Step (c) removing the solvent from the solution.

The solvent(s) may be removed by techniques known in art which includes but are not limited to: distillation, evaporation, oven drying, tray drying, rotational drying (such as the Buchi Rotavapor), spray drying, freeze-drying, fluid bed drying, flash drying, spin flash drying, agitated thin film drying, and the like.

The solvent(s) can be removed from the solution by distillation under vacuum. The solvent can be distilled under reduced pressure maintained at about 1 to about 100 mbar, for example from 10 to 30 mbar. The distillation can be conducted at temperatures from about 30° C. to about 125° C., to dryness.

Step (d) involves optionally adding isopropyl alcohol to the obtained reaction mass.

Isopropyl alcohol is optionally added to the reaction mass obtained in step (c) in an amount sufficient to form a homogeneous solution at or below the reflux temperature of the solvent.

Step (e) involves optionally removing the solvent from the solution to provide the desired amorphous prasugrel hydrochloride.

The solvent can be also removed from the solution by techniques known in art which includes but are not limited to: distillation, evaporation, oven drying, tray drying, rotational drying (such as the Buchi Rotavapor), spray drying, freeze-drying, fluid bed drying, flash drying, spin flash drying, agitated thin film drying, and the like.

Suitable temperatures for removing the solvent can be about −30° C. to about 60° C. with or without vacuum.

Step (f) involves drying the product obtained in Step (e).

The solid may be further dried. Drying can be suitably carried out in a tray dryer, vacuum oven, Rotavapor, air oven, fluidized bed drier, spin flash dryer, flash dryer and the like. The drying may be carried out at temperatures of about 35° C. to about 70° C. The drying can be carried out for any time periods necessary for obtaining a desired purity, such as, for example, from about 1 to about 25 hours, or longer.

The present invention includes processes for the preparation of an amorphous form of prasugrel hydrochloride comprising at least one of the steps of:

(a) providing a solution of prasugrel hydrochloride in a solvent;

(b) removing the solvent from the solution; and

(c) drying the product obtained in Step (b).

Step (a) involves providing a solution of prasugrel free base in a solvent.

The solution of prasugrel hydrochloride may be provided by the dissolution or making a slurry of prasugrel free base in a solvent, or it may be obtained from the reaction mass of a synthesis step. Any form of prasugrel hydrochloride, such as, for example, anhydrous crystalline forms, amorphous, or mixtures of crystalline and amorphous forms of prasugrel in any proportions, obtained by any method, is acceptable for forming the solution.

The solution of prasugrel hydrochloride may be prepared at room temperature to the reflux temperature of the solvent, depending on the quantity of solvent used.

The solvents which may be used for providing a solution may be from the various classes of solvents, such as, for example, alcoholic solvents, ketones, esters, ethers, halogenated solvents, hydrocarbons, nitriles, water aprotic polar solvents or mixtures thereof. Alcohol solvents include, for example, methanol, ethanol, denatured spirit, n-propanol, isopropanol, n-butanol, isobutanol, and t-butanol and the like. Ketonic solvents include, for example, acetone, propanone, 2-butanone and the like. Halogenated solvents include, for example, dichloromethane, 1,2-dichloroethane, chloroform, and the like. Ester solvents include, for example, ethyl acetate, n-propyl acetate, isopropyl acetate and n-butyl acetate and the like. Ether solvents include, for example, dimethyl ether, diethylether, methyl tertiary-butyl ether, ethyl methyl ether, diisopropyl ether, tetrahydrofuran, dioxane and the like. The hydrocarbon may be any solvent from this class, such as, for example, toluene, xylene, cyclohexane, n-hexane, n-heptane, and the like. The nitrile solvents may include acetonitrile, propionitrile and the like, or mixtures thereof or their aqueous combinations in various ratios without limitation.

The undissolved particles may be removed suitably by filtration, centrifugation, decantation, and other techniques. The solution may be filtered by passing through paper, glass fiber, or other membrane material, or a clarifying agent, such as, for example, celite. Depending upon the equipment used and the concentration and temperature of the solution, the filtration apparatus may need to be preheated to avoid premature crystallization.

Step (b) involves removing the solvent from the solution.

The solvent may be removed by techniques known in art which include but are not limited to: distillation, evaporation, oven drying, tray drying, rotational drying (such as the Buchi Rotavapor), spray drying, freeze-drying, fluid bed drying, flash drying, spin flash drying, agitated thin film drying, and the like.

The solvent can be removed from the solution by distillation under vacuum. The solvent can be distilled under reduced pressure maintained at about 1 to about 100 mbar, for example from 10 to 30 mbar. The distillation can be conducted at temperatures from about 30° C. to about 125° C., to dryness.

Step (c) involves drying the product obtained in Step (b).

The solid may be further dried. Drying can be suitably carried out in a tray dryer, vacuum oven, Buchi Rotavapor, air oven, fluidized bed drier, spin flash dryer, flash dryer and the like. The drying may be carried out at temperatures of about 35° C. to about 70° C. The drying can be carried out for any time periods necessary for obtaining a desired purity, such as, for example, from about 1 to about 25 hours, or longer.

The present invention includes prasugrel hydrochloride crystalline Forms C, D, and E and amorphous form, characterized by having substantial purity. The crystalline Forms C, D and E and amorphous form may contain less than about 0.5%, or less than about 0.1%, of the process related impurities as determined by high performance liquid chromatography (HPLC).

An example of a HPLC method that can be used for the analysis of prasugrel includes a Cadenza CD C-18, 150×4.6 mm, 3 μm or equivalent column. Additional method parameters are given in Table 1.

TABLE 1

| Flow rate | 1.2 ml/minute | | |
|---|---|---|---|
| Elution | gradient | | |
| Wavelength | 210 nm | | |
| Injection | 10 μl | | |
| volume | | | |
| Column oven | ambient | | |
| temperature | | | |
| Run time | 65 minutes | | |
| Diluent | acetonitrile | | |
| Sample | 1.0 mg/ml | | |
| concentration | | | |
| Buffer | dissolve 2.72 g of KH₂PO₄ in 1000 ml of Milli-q water, | | |
| preparation | add 1.5 ml of triethylamine and adjust pH to 6.0 with | | |
| | dilute orthophosphoric acid. | | |
| Mobile phase | Mobile phase A: mix buffer and acetonitrile in the | | |
| | volume ratio 80:20. | | |
| | Mobile phase B: mix buffer and acetonitrile in the | | |
| | volume ratio 20:80. | | |
| Gradient | Minutes | Mobile Phase A | Mobile Phase B |
| program | 0 | 70 | 30 |
| | 5 | 70 | 30 |
| | 25 | 40 | 60 |
| | 35 | 20 | 80 |
| | 45 | 10 | 90 |
| | 55 | 10 | 90 |
| | 60 | 50 | 50 |
| | 65 | 70 | 30 |

The present invention includes processes for the conversion of a weak acid addition salt of prasugrel to a strong acid addition salt of prasugrel. The weak acid addition salt can be any of the organic salts like formate, acetate, tartarate, maleate, succinate and besylate and the strong acid addition salt is a hydrochloride, hydrogen sulfate, hydrobromide, preferably hydrochloride.

The process of conversion of weak acid addition salt of prasugrel to a strong acid addition salt of prasugrel involves providing a solution of weak acid addition salt of prasugrel in a suitable organic solvent, adding a source of strong acid (viz, hydrogen chloride), optionally seeding with strong acid salt of prasugrel, collecting the precipitated solid and optionally drying the solid.

The organic solvents that may be used for this step may be organic solvents from the various classes of solvents, such as, for example, alcoholic solvents, ketones, esters, ethers, halogenated solvents, hydrocarbons, nitriles, water aprotic polar solvents or mixtures thereof. These include: alcohol solvents, such as, for example, methanol, ethanol, denatured spirits, n-propanol, isopropanol, n-butanol, isobutanol, and t-butanol and the like; ketonic solvents, such as, for example, acetone, propanone, 2-butanone and the like; halogenated solvents, such as, for example, dichloromethane, 1,2-dichloroethane, chloroform, and the like; ester solvents, such as, for example, ethyl acetate, n-propyl acetate, isopropyl acetate and n-butyl acetate and the like; ether solvents, such as, for example, dimethyl ether, diethylether, methyl tertiary-butyl ether, ethyl methyl ether, diisopropyl ether, tetrahydrofuran, dioxane and the like. The hydrocarbon may be any solvent from this class, such as, for example, toluene, xylene, cyclohexane, n-hexane, n-heptane and the like. The nitrile solvents may include acetonitrile, propionitrile and the like; or mixtures thereof or their aqueous combinations in various ratios without limitation.

Suitable sources of hydrogen chloride include, and are not limited to, aqueous hydrogen chloride (5%-36%) or solutions of hydrogen chloride in an organic solvent, such as, for example, methanol, ethanol, ethyl acetate, isopropyl alcohol, and acetone, or dry hydrogen chloride gas, or mixtures thereof.

The reaction mass may be maintained at temperatures ranging from −10° C. to 50° C. The reaction mass may be maintained for about 30 minutes to 10 hours, or longer.

The products may be isolated and dried using the techniques described above.

The process of the conversion of weak addition salts of prasugrel to prasugrel hydrochloride can be to any desired polymorph, such as crystalline Forms A, B1, B2, C, D, or E, or an amorphous form, of prasugrel hydrochloride.

The prasugrel or its salts can optionally be milled to get a desired particle size. Milling or micronization can be performed prior to drying, or after the completion of drying of the product. The milling operation reduces the size of particles and increases surface area of particles by colliding particles with each other at high velocities.

The present invention includes pharmaceutical compositions comprising a therapeutically effective amount of substantially pure prasugrel or its pharmaceutically acceptable salt and at least one pharmaceutically acceptable excipient.

Prasugrel or its salts obtained by the processes of the present invention can be formulated into solid pharmaceutical compositions for oral administration in the form of capsules, tablets, pills, powders or granules. In these compositions, the active product is combined with one or more pharmaceutically acceptable excipients. The drug substance can be formulated as liquid compositions for oral administration including for example solutions, suspensions, syrups, elixirs and emulsions, containing solvents or vehicles, such as, for example, water, sorbitol, glycerine, propylene glycol or liquid paraffin.

The compositions for parenteral administration can be suspensions, emulsions or aqueous or non-aqueous, sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g., using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable carriers including and are not limited to diluents, such as, for example, starch, pregelatinized starch, lactose, powdered celluloses, microcrystalline celluloses, dicalcium phosphate, tricalcium phosphate, mannitol, sorbitol, sugar and the like; binders, such as, for example, acacia, guar gum, tragacanth, gelatin, polyvinylpyrrolidones, hydroxypropyl celluloses, hydroxypropyl methylcelluloses, pregelatinized starches, and the like; disintegrants, such as, for example, starches, sodium starch glycolate, pregelatinized starches, crospovidones, croscarmellose sodiums, colloidal silicon dioxide and the like; lubricants, such as, for example, stearic acid, magnesium stearate, zinc stearate and the like; glidants, such as, for example, colloidal silicon dioxide and the like; solubility or wetting enhancers, such as, for example, anionic or cationic or neutral surfactants, complex forming agents, such as, for example, various grades of cyclodextrins and resins; release rate controlling agents, such as, for example, hydroxypropyl celluloses, hydroxymethyl celluloses, hydroxypropyl methylcelluloses, ethyl celluloses, methyl celluloses, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

Certain specific aspects and embodiments of the present invention will be explained in more detail with reference to the following examples, which are provided by way of illustration only and should not be construed as limiting the scope of the invention in any manner.

### EXAMPLE 1: PREPARATION OF 5-TRITYL-4,5,6,7-TETRAHYDRO-THIENO[3,2-c]PYRIDINE

4,5,6,7-Tetrahydrothieno[3,2-c]pyridine hydrochloride (100 g), water (500 mL), aqueous ammonium hydroxide solution (40 mL), and dichloromethane (500 mL) are charged into a round bottom flask and stirred for 30 minutes at 23° C. The organic layer and aqueous layer are separated. The aqueous layer is extracted with dichloromethane (100 mL). The organic layers are combined and dried over sodium sulfate. The resultant organic layer and triethylamine (88.0 mL) are charged into another round bottom flask. The solution of trityl chloride (158.0 g of trityl chloride in 250 mL dichloromethane) is added dropwise at 23° C. over a period of 1 hour. Water (500 mL) is added to the reaction mixture and then two layers are separated. Organic layer is dried over sodium sulfate and filtered. The obtained filtrate is concentrated completely under vacuum at 42° C. Methanol (500 mL) is charged to the obtained residue and it is stirred for 40 minutes at 25° C. The obtained suspension is filtered and washed with methanol (200 mL). The product is dried at 51° C. for 18 hours to afford 162.5 g of title compound.

Purity: 68.916% by HPLC.

### EXAMPLE 2: PREPARATION OF 5-TRITYL-5,6,7,7a-TETRAHYDRO-4H-THIENO[3,2-c]PYRIDONE

5-Trityl-4,5,6,7-tetrahydro-thieno[3,2-c]pyridine (100 g) and tetrahydrofuran (1000 mL) are charged into a round bottom flask. n-Butyl lithium (1.6 molar solution in hexane; 246 mL) is added dropwise at 0° C. for 1 hour. The mixture is stirred at 10° C. for 1 hour 30 minutes. Tri-n-butyl borate (156.0 mL) and tetrahydrofuran (200 mL) are added to the obtained reaction mixture at −5° C. dropwise for 1 hour. The obtained reaction mixture is stirred for 1 hour at 10° C. and then cooled to −10° C. Hydrogen peroxide (30%, 74 mL) is added to the reaction mixture dropwise for a period of 5 minutes. The reaction mixture is allowed to rise to a temperature of 30° C. Water (1000 mL) is added to the reaction mixture and stirred for 35 minutes. The reaction mixture is extracted with ethyl acetate (1200 mL). The organic layer is washed with water (2×500 mL) and dried over sodium sulfate. The combined organic layer is concentrated completely at 45° C. Diisopropyl ether (600 mL) is added to the reaction residue and stirred at 22° C. for 20 minutes. The obtained suspension is filtered and the solid is washed with diisopropyl ether (200 mL). The obtained solid is dried at 50° C. for 3 hours to afford 75.9 g of title compound.

Purity: 92% by HPLC

### EXAMPLE 3: PREPARATION OF 5,6,7,7a-TETRAHYDRO-THIENO[3,2-c]PYRIDIN 2(4H)-ONE HYDROCHLORIDE

5-Triphenylmethyl 5,6,7,7a-tetrahydro 4H-thieno[3,2-c]pyridone (80.0 g), acetone (1200 mL), and hydrochloric acid (20 mL) are charged into a round bottom flask. The resulting mixture is heated to reflux (56° C.) and stirred for 3 hours at reflux. The reaction mixture is cooled to 30° C. The obtained suspension is filtered and the solid is washed with acetone (100 mL). The solid is dried at 60° C. for 3 hours to afford 37.5 g of title compound.

Purity: 97.74% by HPLC.

### EXAMPLE 4: PREPARATION OF CYCLOPROPYL-2-FLUOROBENZYL KETONE

Magnesium powder (15.2 g), anhydrous diethyl ether (330 mL) and iodine (0.2 g) are charged into a round bottom flask. A solution of 2-fluoro benzyl bromide (100 g) in diethyl ether (330 mL) is added over a period of 1 hour, 45 minutes and stirred at 30° C. for 1 hour. A solution of cyclopropyl cyanide (46 g) in diethyl ether (330 mL) is added to the above obtained reaction mixture over a period of 1 hour and stirred for 2 hours at 30° C. The reaction mixture is quenched with saturated ammonium chloride solution (500 mL). The layers are separated. The organic layer is washed with saturated bicarbonate solution (500 mL), saturated aqueous sodium chloride solution (500 mL) and the organic layer is dried over sodium sulfate. The organic solvent is concentrated completely at a temperature of 55° C. to afford 82.6 g of crude product.

Purity: 81.9% by HPLC.

### EXAMPLE 5: PREPARATION OF 2-FLUORO-α-CYCLOPROPYL CARBONYL BROMIDE

Cyclopropyl-2-fluorobenzyl ketone (50 g), carbon tetrachloride (1000 mL), N-bromo succinamide (60 g), AIBN (3 g) and PTSA (1.5 g) are charged into a round bottom flask with stirring. The reaction mixture is heated to reflux (75° C.) and stirred for 3 hours. The reaction mixture is cooled to 15° C. and then the suspension is filtered. The filtrate is washed with 5% sodium bisulfate solution (2×250 mL) and then the obtained organic layer is dried over sodium sulfate. The resultant organic layer is concentrated completely under vacuum at 50° C. to provide crude product. Hexane (300 mL) is charged to the obtained crude mass and stirred for 10 minutes followed by decantation of n-hexane. The obtained decanted n-hexane layer is distilled completely at 36° C. to afford 55.0 g of the title compound.

Purity: 73.6% by HPLC.

### EXAMPLE 6: PREPARATION OF 5-[2-CYCLOPROPYL-1-(2-FLUOROPHENYL)-2-OXOETHYL]5,6,7,7a-TETRAHYDRO-THIENO[3,2-c]PYRIDIN-2(4H)-ONE

α-Cyclopropyl carbonyl 2-fluorobenzyl bromide (50.0 g) and dimethyl formamaide (50 mL) are charged into a round bottom flask. Potassium carbonate (50 g) is charged with stirring to the above suspension. The reaction mixture is cooled to 5° C. A solution of 5,6,7,7a-tetrahydro-thieno[3,2-c]pyridine-2-(4H)-one hydrochloride (45.0 g) in dimethylformamide (50 mL) is added over 25 minutes at 5° C. and stirred for 30 minutes. The reaction mixture is further stirred for 1 hour, 45 minutes at 25° C. The reaction mixture is decomposed by adding chilled water (500 mL) and then water (300 mL) is decanted from the reaction mixture. Ethyl acetate (500 mL) is charged to the obtained reaction mixture. The layers are separated. The obtained ethyl acetate layer is washed with 5% saturated aqueous sodium chloride solution (2×100 mL), and then dried over sodium sulfate. The organic solvent is concentrated completely under reduced pressure at 52° C. The reaction crude is extracted into ethyl acetate (300 mL) and the obtained ethyl acetate layer is concentrated completely at 55° C. to afford 20.2 g of the title compound.

### EXAMPLE 7: PREPARATION OF 2-ACETOXY-5-(A-CYCLOPROPYL CARBONYL-2-FLUOROBENZYL)-4,5,6,7-TETRAHYDROTHIENO[3,2-c]PYRIDINE (FORMULA I)

5-[2-Cyclopropyl-1-(2-fluorophenyl)-2-oxoethyl]5,6,7,7a-tetrahydro-thieno[3,2-c]pyridin-2(4h)-one (20.0 g) and dimethylformamide (75 mL) are charged into a round bottom flask. The reaction mixture is cooled to 10° C. and then acetic anhydride (37.5 mL) is added to the obtained reaction solution. Sodium hydride (60% dispersion in mineral oil, 3.0 g) is added to the reaction mixture at 10° C. over a period of 25 minutes, and then the mixture is stirred at a temperature of 28° C. for 1 hour 45 minutes. Saturated ammonium chloride (100 mL) is added to the obtained reaction mixture. The mixture is extracted with ethyl acetate (2×100 mL) and the separated organic layer is washed with saturated sodium chloride solution (2×100 mL). The obtained organic layer is concentrated completely under reduced pressure at 50° C. The crude product is dissolved in diisopropyl ether (50 mL) at 30° C. and it is kept overnight. The obtained suspension is filtered and the solid is washed with 10 mL of diisopropyl ether. The solid is dried at 50° C. for 2 hours to afford 6.2 g of the title compound.

Purity: 95.46% by HPLC.

The obtained compound (6.0 g) and methanol (60 mL) are charged into a round bottom flask. The mixture is heated to reflux (65° C.) and stirred for 30 minutes. The obtained solution is allowed to cool to a temperature of 30° C. and filtered. The solid is washed with methanol (12 mL) and dried for 2 hours at 55° C. to afford 4.8 g of title compound.

Purity: 98.4% by HPLC.

### EXAMPLE 8: PREPARATION OF ACETIC ACID 5-TRITYL-4,5,6,7-TETRAHYDRO-THIENO[3,2-c]PYRIDIN-2-YL ESTER

5-Trityl 5,6,7,7a-tetrahydro-4H-thieno[3,2-c]pyridone (50.0 g) and dimethylformamide (500 mL) are charged into a round bottom flask. Acetic anhydride (100 mL) is added dropwise at 5° C. over a period of 15 to 30 minutes. Sodium hydride (60% dispersion in mineral oil, 7.5 g) is added to the reaction mixture at a temperature of 0° C. to 10° C. The reaction mixture is maintained for 1 to 2 hours at a temperature of 25° C. to 35° C. Ethyl acetate (500 mL) is added to the reaction mixture and then the separated organic layer is washed with saturated sodium chloride solution (3×50 mL). The organic layer is concentrated completely under vacuum at a temperature of 35° C. to 45° C. and the obtained residue is dissolved in methanol (500 mL). To the resultant organic layer, water (250 mL) is added at a temperature of 25° C. to 35° C. and maintained at an ambient temperature for 30 minutes. The solid is filtered and dried under vacuum at a temperature of 40° C. to 50° C. to afford 60 g of the title compound.

Purity: 89.4% by HPLC.

### EXAMPLE 9: PREPARATION OF ACETIC ACID 4,5,6,7-TETRAHYDRO-THIENO[3,2-c]PYRIDIN-2-YL ESTER HYDROCHLORIDE

Acetic acid 5-trityl-4,5,6,7-tetrahydro-thieno[3,2-c]pyridin-2-yl ester (66.0 g) and acetone (660 mL) are charged into a round bottom flak. Aqueous hydrochloric acid (18.2 g) and acetone (20.0 mL) mixture is added at a temperature of 0° C. to 10° C. The reaction mixture is allowed to warm to the temperature of 25° C. to 35° C. and then maintained at that temperature for 2 to 3 hours. The precipitated solid is filtered and washed with acetone (50 mL). The product is dried under vacuum at a temperature of 45° C. to 50° C. to afford 25.0 g of the title compound.

Purity: 82.23% by HPLC.

### EXAMPLE 10: PREPARATION OF 2-ACETOXY-5-(A-CYCLOPROPYL CARBONYL-2-FLUOROBENZYL)-4,5,6,7-TETRAHYDROTH I ENO[3,2-c]PYRIDINE (FORMULA I)

Acetic acid 4,5,6,7-tetrahydro-thieno[3,2-c]pyridin-2-yl ester hydrochloride (54.0 g), dichloromethane (750 mL) and diisopropylethylamine (94.6 mL) are charged into a round bottom flask. A solution of 2-fluoro-α-cyclopropylcarbonyl bromide (68.0 g of 2-fluoro-α-cyclopropylcarbonyl bromide in 250 mL of dichloromethane) is added to the reaction mixture dropwise at a temperature of 25° C. to 30° C. and maintained at that temperature for 2 to 3 hours. Water (500 mL) is added to the reaction mixture and layers are separated. Organic layer is washed with water (3×100 mL) and then dried over sodium sulfate followed by evaporation under reduced pressure at a temperature of 35° C. to 45° C. The resulting residue is subjected to silica gel column chromatography, using a 20:80 mixture of ethyl acetate and hexane by volume as the eluent, to give a yellow oil. Diisopropyl ether (200 mL) is added to the obtained oil and stirred for 10 minutes at a temperature of 25° C. to 35° C. The obtained suspension is filtered and the solid is dried at a temperature of 50° C. to 55° C. for 1 to 2 hours to afford the title compound (9.0 g).

Purity: 74% by HPLC.

The above obtained product (4.1 g) and methanol (41 mL) are charged into a round bottom flask and heated to reflux temperature. The mixture is stirred for 30 minutes and then allowed to cool to a temperature of 25° C. to 35° C. The mixture is cooled to a temperature of 10° C. to 20° C. and stirred for 45 minutes. The obtained suspension is filtered and washed with methanol (10 mL). Finally, the solid is dried at a temperature of 45° C. to 55° C. for 2 hours to afford 3.4 g of title compound.

Purity: 99.2% by HPLC.

### EXAMPLE 11: PREPARATION OF PRASUGREL HYDROCHLORIDE AMORPHOUS FORM

To a solution of prasugrel free base (500 mg) in methylene dichloride (10 mL) in a clean and dry round bottom flask, a mixture of methanol (2 mL) and aqueous hydrochloric acid (135 mg) is added at 25° C. The reaction mixture is heated to 40° C. and then the solvent is distilled off. To the residue, isopropyl alcohol (10 mL) is added and then the solvent is distilled off completely. The obtained solid is dried for 4 hours at 40° C. to afford 510 mg of the title compound.

### EXAMPLE 12: ALTERNATE PROCESS FOR THE PREPARATION OF PRASUGREL HYDROCHLORIDE AMORPHOUS FORM

Prasugrel hydrochloride (500 mg) is dissolved in a mixture of isopropyl alcohol (10 mL) and water (0.1 mL). The solvent is evaporated at 30° C. under vacuum. The solid is dried at the same temperature for 3 hours to afford 400 mg of the title compound.

### EXAMPLE 13: PREPARATION OF PRASUGREL HYDROCHLORIDE CRYSTALLINE FORM C

To a mixture of prasugrel free base (1000 mg) and 2-butanol (20 mL) in a round bottom flask at 40° C., aqueous hydrochloric acid (271 mg) is added and the content is stirred for 60 minutes. The precipitated solid is filtered and dried at 60° C. for 2 hours to afford 800 mg of the title compound.

### EXAMPLE 14: ALTERNATE PROCESS FOR THE PREPARATION OF PRASUGREL HYDROCHLORIDE CRYSTALLINE FORM C

To a solution of prasugrel (500 mg) and 2-butanol (10 mL) in a round bottom flask at 28° C., a mixture of 2-butanol (2.0 mL) and aqueous hydrochloric acid (135 mg) is added and it is stirred for 15 minutes. The reaction mixture is seeded with 2.0 mg of crystalline Form C crystal and stirred for 60 minutes. The separated solid is filtered, washed with 2-butanol (5 mL) and dried at 60° C. for 4 hours to afford 400 mg of the title compound.

### EXAMPLE 15: PREPARATION OF PRASUGREL HYDROCHLORIDE CRYSTALLINE FORM D

A mixture of prasugrel free base (500 mg) and isopropyl alcohol (10 mL) in a round bottom flask are heated to 40° C. for 15 minutes. The pH of the reaction mass is adjusted to 2 using a solution of isopropyl alcohol hydrochloride (150 mg). The contents are stirred for 60 minutes and then filtered. The wet cake is then dried at 60° C. for 2 hours to afford 400 mg of the title compound.

### EXAMPLE 16: PREPARATION OF PRASUGREL HYDROCHLORIDE CRYSTALLINE FORM E

A solution of prasugrel free base (500 mg) and ethyl acetate (10 mL) in a round bottom flask are heated to 40° C. for 15 minutes. The pH of the reaction mass is adjusted to 2.0 using a solution of ethyl acetate hydrochloride (150 mg). The contents are stirred for 60 minutes and filtered. The wet cake is then dried at 60° C. for 2 hours to afford 400 mg of the title compound.

### EXAMPLE 17: PREPARATION OF PRASUGREL HYDROCHLORIDE CRYSTALLINE FORM B2

To a solution of prasugrel maleate (1000 mg) and acetone (10 mL) in a round bottom flask at 25° C., a mixture of aqueous hydrochloric acid (0.25 mL) and acetone (5 mL) is added and it is stirred for 90 minutes. The solution is seeded with 2 mg of crystalline Form B2 crystal and stirred for 60 minutes. The separated solid is filtered and washed with acetone (5 mL). The wet cake is dried at 55° C. for 4 hours to afford 610 mg of the title compound.

### EXAMPLE 18: PREPARATION OF PRASUGREL HYDROCHLORIDE CRYSTALLINE FORM B1

To a solution of prasugrel free base (500 mg) and acetone (10 mL) in a round bottom flask at 40° C., hydrogen chloride gas purged in acetone (1.95 g) is added and it is stirred at the same temperature for two hours. The precipitated solid is filtered and dried at 60° C. for 3 hours to afford 400 mg of the title compound.

1. A process for preparing prasugrel of Formula I 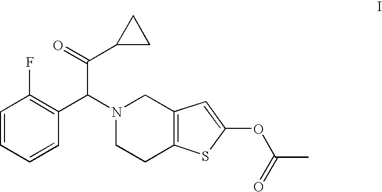
comprising reacting a compound of Formula II 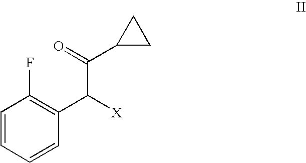
wherein X is halogen,
with a compound of Formula III or its acid addition salt 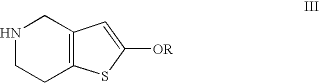
wherein R is acetyl or an hydroxyl-protecting group other than acetyl.

2. The process of claim 1, wherein X is bromine or chlorine.

3. The process of claim 1, wherein R is acetyl.

4. The process of claim 1, wherein the reaction of a compound of Formula II with a compound of Formula III or its salt is carried out in the presence of a base or a solvent, or both a base and a solvent.

5. The process of claim 4, wherein a base is an alkali metal carbonate, an alkaline earth metal carbonate, a hydrogen carbonate, a hydroxide, a oxide, a carboxylate, an alkoxide, a tertiary amine, triethylamine, N,N-diisopropylethyl amine, N,N-diethylethanamine, N-(1-methylethyl)-2-propanamine, 4-ethylmorpholine, 1,4-diazabicyclo[2.2.2]-octane, N-methyl morpholine, pyridine, or any mixtures thereof.

6. The process of claim 4, wherein a solvent is an alcohol, a halogenated hydrocarbon, a ketone, an ether, a hydrocarbon, an aromatic solvent, a nitrile, dimethylsulfoxide, N,N-dimethylformamide, N,N-dimethylacetamide, pyridine, 1,3-dimethyl-3,4,5,6-tetrahydro-2(1H)-pyrimidinone, 1,3-dimethyl-2-imidazolidinone, 1,1,3,3-tetramethylurea, 1-methyl-2-pyrrolidinone, nitrobenzene, water, and any mixtures thereof.

7. A process for preparing prasugrel comprising:
(a) oxidizing 5-trityl-4,5,6,7-tetrahydrothieno[3,2-c]pyridine of Formula VI, 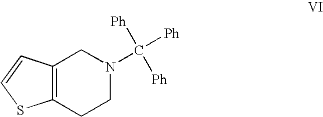
where Ph is phenyl, to provide 5-trityl-5,6,7,7a-tetrahydrothieno[3,2-c]pyridone of Formula VII; 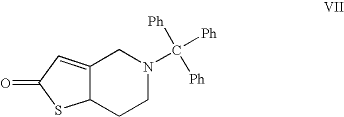
(b) reacting the compound of Formula VII with an acetylating agent in to produce 5-trityl-4,5,6,7-tetrahydrothieno[3,2-c]pyridin-2-yl ester of Formula VIII-A; 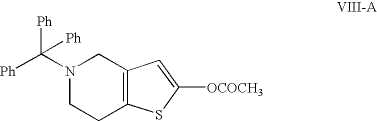
(c) removing the trityl group from the compound of Formula VIII-A to form the compound of Formula III or its acid addition salt; 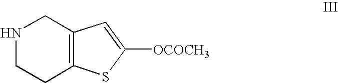
(d) reacting the compound of Formula III with a compound of Formula II, 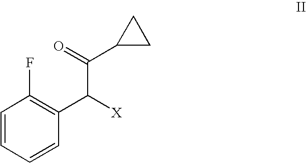
where X is a halogen, to form prasugrel; and
(e) optionally, converting prasugrel into its pharmaceutically acceptable salt.

8. The process of claim 7, wherein an acetylating agent in (b) is acetic anhydride or acetyl chloride.

9. The process of claim 7, wherein a halogen in (d) is bromine or chlorine.

10. The process of claim 7, wherein the reaction of a compound of Formula II with a compound of Formula III or its salt is carried out in the presence of a base or a solvent, or both a base and a solvent.

11. The process of claim 10, wherein a base is an alkali metal carbonate, an alkaline earth metal carbonate, a hydrogen carbonate, a hydroxide, an oxide, a carboxylate, an alkoxide, a tertiary amine, triethylamine, N,N-diisopropylethyl amine, N,N-diethylethanamine, N-(1-methylethyl)-2-propanamine, 4-ethylmorpholine, 1,4-diazabicyclo[2.2.2]-octane, N-methyl morpholine, pyridine, or any mixtures thereof.

12. The process of claim 10, wherein a solvent is an alcohol, a halogenated hydrocarbon, a ketone, an ether, a hydrocarbon, an aromatic solvent, a nitrile, dimethylsulfoxide, N,N-dimethylformamide, N,N-dimethylacetamide, pyridine, 1,3-dimethyl-3,4,5,6-tetrahydro-2(1H)-pyrimidinone, 1,3-dimethyl-2-imidazolidinone, 1,1,3,3-tetramethylurea, 1-methyl-2-pyrrolidinone, nitrobenzene, water, or any mixtures thereof.

13. A process for preparing prasugrel or its pharmaceutically acceptable salt comprising:
(a) treating 5-trityl-5,6,7,7a-tetrahydrothieno[3,2-c]pyridin-2(4H)-one of Formula VII, 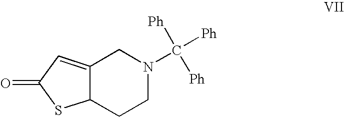
with hydrochloric acid to provide the compound of Formula V or its salt; 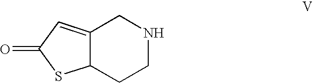
(b) condensing 2-fluoro-α-cyclopropylcarbonyl bromide of Formula II, 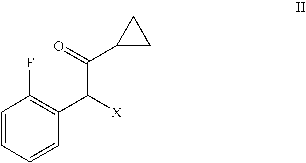
wherein X is halogen, with the compound of Formula V or its salt or tautomer thereof in the presence of a base to form 5-[2-cyclopropyl-1-(2-fluorophenyl)-2-oxoethyl]-5,6,7,7a-tetrahydrothieno[3,2-c]pyridin-2(4H)-one of Formula IX; 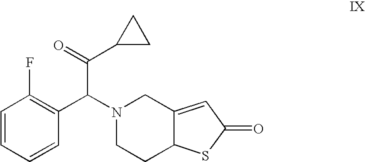
(c) reacting the compound of Formula IX with an acetylating agent to afford prasugrel; and
(d) optionally, converting prasugrel into its pharmaceutically acceptable salt.

14. The process of claim 13, wherein an acetylating agent in (b) is acetic anhydride or acetyl chloride.

15. The process of claim 13, wherein a halogen in (d) is bromine or chlorine.

16. The process of claim 13, wherein the reaction of a compound of Formula II with the compound of Formula III or its salt is carried out in presence of a base or a solvent, or both a base and a solvent.

17. The process of claim 16, wherein a base is potassium carbonate, sodium carbonate, cesium carbonate, potassium t-butoxide, sodium t-butoxide, potassium hydroxide, sodium hydroxide, potassium bicarbonate, triethylamine, N,N-diisopropylethyl amine, or mixtures thereof.

18. The process of claim 16, wherein the solvent is N,N-dimethylformamide, dimethylsulfoxide, N,N-dimethylacetamide, dichloromethane, ethylenedichloride, chloroform, or mixtures thereof.

19. A crystalline Form C of prasugrel hydrochloride characterized by an XRD pattern having peaks at diffraction angles 2-theta of about 8, 8.4, 11.9, 12.5, 12.8, 13.2, 15.2, 15.4, 18.5, 20.4, 20.7, 23.4, 24.4, 24.6, 25.2, 25.8, 26.6, and 27.3, ±0.2 degrees.

20. A crystalline Form D of prasugrel hydrochloride characterized by an XRD pattern having peaks at diffraction angles 2-theta of about 8.1, 13.6, 14.6, 16.2, 20.7, 22.1, 24.5, 25.5, 25.9, 27.4, 30.1, and 32.8, ±0.2 degrees.

21. A crystalline Form E of prasugrel hydrochloride characterized by an XRD pattern having peaks at diffraction angles 2-theta of about 8, 8.4, 12.9, 13.5, 14.5, 16.1, 20.6, 21.4, 22, 25.6, 25.8, and 27.3, ±0.2 degrees.

22. Amorphous prasugrel hydrochloride.

23. A compound of Formula III or Formula VIII 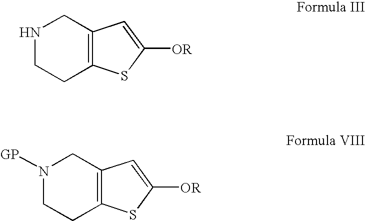
wherein R is a hydroxyl-protecting group and PG is a nitrogen-protecting group.

24. A compound of claim 23, wherein R is acetyl and PG is trityl or t-butyloxycarbonyl.
